(19) United States
(12) Patent Application Publication
(10) Pub. No.: US 2005/0079131 A1
(43) Pub. Date: Apr. 14, 2005

(54) Emulsion particles for imaging and therapy and methods of use thereof

(76) Inventors: Gregory M. Lanza, St. Louis, MO (US); Samuel A. Wickline, St. Louis, MO (US)

Correspondence Address: MORRISON & FOERSTER LLP, 3811 VALLEY CENTRE DRIVE, SUITE 500, SAN DIEGO, CA, 92130-2332, US

(21) Appl. No.: 10/913,846

(22) Filed: Aug. 6, 2004

Related U.S. Application Data
(60) Provisional application No. 60/493,492, filed on Aug. 8, 2003.

(51) Int. Cl. A61K 51/00; A61K 49/00

(52) U.S. Cl. 424/1.11; 424/9.36

(57) ABSTRACT

Emulsions preferably of nanoparticles formed from oil compounds coupled to a high Z number atom, said particles coated with a lipid/surfactant coating. The nanoparticles are made specific to targeted cells or tissues by coupling said nanoparticles to a ligand specific for the target cells or tissues. The nanoparticles may further include biologically active agents, radionuclides and/or other imaging agents.

### CROSS-REFERENCE TO RELATED APPLICATIONS

This application claims benefit under 35 U.S.C. § 119(e) to provisional application 60/493,492 filed 8 Aug. 2003, which is incorporated herein by reference.

### TECHNICAL FIELD

This invention relates generally to nanoparticle-containing emulsions for use as contrast agents for imaging and/or delivery of a therapeutic agent. It particularly relates to lipid encapsulated emulsions comprising an oil coupled to a high Z number atom and to such emulsions further containing a targeting ligand. It also relates to the making and administration of the emulsions for imaging and/or delivery of a therapeutic agent.

### BACKGROUND OF THE INVENTION

Molecular imaging can enhance the utility of traditional clinical imaging by allowing specific detection of molecular markers in tissues using site-targeted contrast agents (Weissleder (1999) Radiology 212:609-614). Three approaches to site-targeted ultrasonic agents have been reported and these are based upon the use of liposomes (Alkan-Onyuksel et al. (1996) J. Pharm. Sci. 85:486-490; Demos et al. (1997) J. Pharm. Sci. 86:167-171; Demos et al. (1999) J. Am. Col. Cardiol. 33:867-875), the use of microbubbles (Mattrey et al. (1984) Am. J. Cardiol. 54:206-210; Unger et al. (1998) Am. J. Cardiol. 81:58G-61G; Villanueva et al. (1998) Circulation 98:1-5; Klibanov et al. (1998) Acad. Radiol. 5S243-S246) or the use of nano-emulsions (Lanza et al. (1996) Circulation 94:3334-3340; Lanza et al. (1998) J. Acoust. Soc. Am. 104:3665-3672; Lanza et al. (1997) Ultrasound Med. Biol. 23:863-870).

The value of nanoparticular compositions composed of perfluorocarbon nanoparticles coated with a surfactant layer to facilitate binding of desired components for imaging of various types is well established. See, for example, U.S. Pat. Nos. 5,690,907, 5,780,010, 5,958,371 and 5,989,520; PCT publication WO 02/060524; and Lanza et al., 1998, and Lanza et al., 1997. These documents describe emulsions of perfluorocarbon nanoparticles that are coupled to various targeting agents and to desired components, such as magnetic resonance imaging agents, radionuclides and/or bioactive agents.

Other compositions that have been used for targeted imaging include those disclosed in PCT publications WO 99/58162, WO 00/35488, WO 00/35887 and WO 00/35492.

Although not targeted by inclusion of a specific homing or targeting ligand, iodine-containing fat emulsions have been used as X-ray contrast agents in the imaging of tumors and the like due to uptake of the emulsion particles by cells of the reticuloendothelial system (RES-cells). Through this passive targeting in which these emulsions are taken up by normal clearance organs, the liver and spleen with large quantities of RES-cells are made more radio-opaque than other tissues. The cells of the liver and spleen that take up the iodine-containing fat emulsions depends on the size and composition of the emulsion. For example, generally, emulsions with mean particle size larger than one micron are taken up cells of the lung, spleen and liver and emulsions with mean particle size of about 0.1 to 0.3 microns penetrate into the space of Disse and are taken up and retained by hepatocytes, in addition to RES-cells. See, for example, U.S. Pat. No. 4,917,880. Also, U.S. Pat. No. 5,445,811, describes X-ray contrast agent emulsions based on lipophilic iodized and/or brominated substances with phospholipid surfactants that have small particle sizes which allow for increased uptake into hepatocytes.

Due to uptake and retention of lipiodol and ethiodol in hepatocellular carcinoma, these iodinated derivatives of poppy seed oil have been used to deliver chemotherapeutic or radiotherapeutic agents to these tumors. See, for example, Yu et al. (2003) Appl. Radiat. Isot. 58:567-573; Kountouras et al. (2002) Hepatogastroenterology 49:1109-1112; Al-Mufti et al. (1999) Br. J. Cancer 79:1665-1671; Bhattacharya et al. (1996) Br. J. Cancer 73:877-881; Bretagne et al. (1988) Radiology 168:547-550; Konno et al. (1983) Eur. J. Cancer Clin. Oncol. 19:1053-1065. The retention of these iodinated oils in tumor cells of the liver suggests that these compounds may be useful drug delivery agents, and well as X-ray contrast agents, for possible treatment and imaging of hepatocellular carcinoma.

There remains a continuing need for developing approaches and compositions that are useful for reaching a variety and/or particular sites and tissues within an individual and that result in an enhanced degree of contrast, specificity and sensitivity for molecular imaging systems and therapeutic agent delivery.

All publications and patent applications cited herein are hereby incorporated by reference in their entirety.

### BRIEF SUMMARY OF THE INVENTION

The invention is directed to compositions and methods for imaging and/or therapeutic agent delivery using an oil-in-water emulsion, wherein the oil-in-water emulsion comprises nanoparticles formed from an oil-like compound coupled to an atom with a Z number above 36 and the nanoparticles are coated with a lipid/surfactant layer and the nanoparticles are coupled to a ligand which binds to a target. In some embodiments, the emulsion further comprises at least one biologically active agent. The use of the emulsions in the context of imaging results in improved image quality and the opportunity for multi-modal imaging and therapeutic agent delivery.

In another aspect, the invention is directed to a method for imaging a target tissue with the emulsion. In another aspect the invention is directed to delivery of a bioactive agent to a target tissue with the emulsion. In one embodiment, the target tissue for imaging and/or agent delivery is cardiovascular-related tissue.

In another aspect, the invention is directed to a method of making an oil-in-water emulsion, wherein the oil-in-water emulsion comprises nanoparticles formed from an oil-like compound coupled to an atom with a Z number above 36 and the nanoparticles are coated with a lipid/surfactant layer and the nanoparticles are coupled to a ligand which binds to a target.

### BRIEF DESCRIPTION OF THE DRAWINGS

FIG. 1 is an image showing two examples of fibrin clots exposed to the non-targeted (upper) and targeted (lower) contrast agents.

### DETAILED DESCRIPTION OF THE INVENTION

The present invention offers targeted emulsions containing an oil coupled to a high Z number atom that provide superior imaging of sites and/or delivery of a therapeutic agent. A targeted emulsion comprising an oil coupled to a high Z number atom provides a greatly improved contrast to noise ratio as compared to non-targeted high Z number atom emulsion control agent and as compared to a targeted emulsion without the high Z number atom. When used alone, the nanoparticle-containing emulsions are useful as contrast agents for X-ray imaging (e.g., computed tomography (CT)), ultrasound imaging and/or delivery of a therapeutic agent. Ancillary reagents may also be associated with the nanoparticles of the emulsions for other forms of imaging, such as, magnetic resonance imaging (MRI), nuclear imaging (e.g., scintigraphy, positron emission tomography (PET) and single photon emission computed tomography (SPECT)), optical or light imaging (e.g., confocal microscopy and fluorescence imaging), magnetotomography and electrical impedance imaging. Incorporation of radionuclides in or on the nanoparticles results in emulsions that can be useful both as diagnostic and therapeutic agents. Accordingly, depending on the type of ancillary reagents incorporated, the emulsions may be used with a combination of imaging. For example, multi-modal imaging may be performed with emulsions including ancillary reagents for MRI, such as the combination of X-ray and MRI imaging. In addition, or alternatively, the emulsion may contain one or more bioactive agents in and/or on the high Z number atom oil core. Accordingly, the nanoparticles of the invention may be used as a diagnostic and/or a therapeutic agent.

Emulsions of the invention contain nanoparticles based on oils coupled to a high Z number atom. The liquid emulsion contains nanoparticles comprised of an oil coupled to a high Z number atom, the oil surrounded by a coating which is composed of a lipid and/or surfactant.

In some instances, the lipid and/or surfactant surrounding coating is able to couple directly to a targeting moiety or can entrap an intermediate component which is covalently coupled to the targeting moiety, optionally through a linker, or may contain a non-specific coupling agent such as biotin. Alternatively, the coating may be cationic or anionic so that targeting agents can be electrostatically adsorbed to the surface. For example, the coating may be cationic so that negatively charged targeting agents such as nucleic acids, in general, or aptamers, in particular, can be adsorbed to the surface.

In some embodiments, the nanoparticles may contain associated with their surface at least one “ancillary agent” useful in imaging and/or therapy including, but not limited to, a radionuclide, a contrast agent for MRI or for PET imaging, a fluorophore or infrared agent for optical imaging, and/or a biologically active compound. The nanoparticles themselves can serve as contrast agents for X-ray (e.g., CT) and ultrasound imaging.

In some embodiments, the emulsions may be modified to incorporate therapeutic agents including, but not limited to, bioactive, radioactive, chemotherapeutic and/or genetic agents, for use as a therapeutic agent as well as a diagnostic agent. The therapeutic agents of such emulsions may be on or attached at the surface of the nanoparticles or within the high Z number atom oil core of the nanoparticles.

The invention also provides methods of using the emulsions in a variety of applications including in vivo, ex vivo, in situ and in vitro applications. The methods include single- or multi-modal imaging and/or therapy methods.

Thus, targeted emulsions that incorporate at least one therapeutic agent are particularly useful for the treatment of a disease or disorder that has improved risk/benefit profiles when applied specifically to selected cells, tissues and/or organs. Site-directed, lipid encapsulated emulsions provide an opportunity to deliver therapeutic agents with enhanced efficiency to targeted tissues through a form of therapeutic agent transfer to target cells referred to as contact facilitated delivery. Contact facilitated delivery of therapeutic agents by targeted, lipid-encapsulated emulsions reflects the prolonged association and increased contact of the ligand-bound, lipid-encapsulated particles with the lipid bilayer of the target cell. Without being bound to one particular theory, enhanced intermingling and exchange of lipid components from one lipid surface to the other facilitates the exchange of therapeutic agents in or on the therapeutic emulsion surface to the target cell. Accordingly, targeted cells need not take up the emulsion nor the emulsion need not leak the therapeutic agent for the target cells to receive the therapeutic agent. In comparison, use of emulsions in which a therapeutic agent is carried within the particulate core depend on cell uptake of the emulsion, agent leak from the emulsion or emulsion break-down to deliver the agent to the cell.

Compositions of the Invention

In one embodiment, the preferred emulsion is a nanoparticulate system containing a high Z number atom oil-like compound as a core and an outer coating that is a lipid/surfactant mixture. As such, the nanoparticulate emulsion can serve as a contrast agent, for example, for X-ray and/or ultrasound imaging.

As used herein, the “oil coupled to a high Z number atom” or “high Z number atom oil” or “oil coupled to a high Z number element” or “high Z number element oil” used in the emulsions of the invention includes an oil or oil-like compound that contains at least one atom or element with a Z number above 35 (i.e., from krypton (Kr) onward). Such an atom is referred to herein as a “high Z number atom.” As used herein, “Z number” is equivalent to the number of protons in an atom. In some embodiments the high Z number atom is noncovalently associated with the oil. In some embodiments the high Z number atom is covalently coupled to the oil. In some embodiments, the high Z number element and/or fatty salt of the high Z number element is associated with the oil by simple suspension or dissolution. In some embodiments, the high Z number element may be associated with the oil as a simple suspension or dissolution of a compound containing a high Z number element, a macromolecular structure containing a high Z number atom and/or matrix containing a high Z number element, for example, in a microparticulate or nanparticulate form.

The high Z number atom (or element) of the invention is an atom (or element) with a Z number of 36 or greater, preferably an atom with a Z number of 39 or greater, more preferably an atom with a Z number of 53 or greater. In some embodiments, the atom has a Z number between 36 and 85 (including 36 and 85 and all the Z numbers from 36 to 85). In some instances, the atom has a Z number between 39 and 85 (including 39 and 85 and all the Z numbers from 39 to 85). In some instances, the atom has a Z number between 53 and 85 (including 53 and 85 and all the Z numbers from 53 to 85). In some embodiments, the atom or element with the high Z number includes, but is not limited to, yttrium (Y, Z=39), molybdenum (Mo, Z=42), silver (Ag, Z=47), tin (Sn, Z=50), iodine (I, Z=53) and gold (Au, Z=79). In addition, other high Z number atoms with suitable biocompatibility and radiopacity include zirconium (Zr, Z=40), barium (Ba, Z=56), tantalum (Ta, Z=73), platinum (Pt, Z=78) and bismuth (Bi, Z=83). In some embodiments, the high Z number element associated with the oil is not iodine (I).

The term “radiopacity” refers to a capability of a radiopaque material of being detected by X-rays and conventional radiographic methods, and optionally by other forms of imaging including magnetic resonance imaging and ultrasound imaging.

For use in the emulsions of the invention, the amount of high Z number element in the oil will depend on the Z number of the element. Elements with a higher Z number, e.g., Au, can be used at lower concentrations in the oil, e.g. about 15% w/v, and elements with a lower Z number, e.g., Br, are required at a higher concentration in the oil, e.g., about 50% w/v. For the emulsions, the amount of high Z number element in the oil can range between about 10% and about 60% w/v. In some instances, the amount of element in the oil can be between about 15% and about 50% w/v, between about 20% and about 45% w/v, or between about 25% and about 40% w/v.

As used herein, the term “oil” means a fatty oil or fat that is liquid at the body temperature of the recipient individual or culture temperature of the cells receiving the emulsion. Thus, such an oil will generally melt or at least begin to melt below about 40° C. and preferably below about 35° C. Oils that are liquid at about 25° C. may facilitate injection or other administration of some compositions of this invention.

Any pharmaceutically acceptable oil can be used as an oil coupled to a high Z number atom in the emulsions of the invention. Examples of such oils include, but are not limited to, vitamin A complexes and derivatives, vitamin E complexes and derivatives, poppy seed oil, soybean oil, olive oil, palm oil, teaseed oil, castor oil, sesame oil, grapeseed oil, rape oil, walnut oil, corn oil, kapok oil, rice bran oil, peanut oil, cottonseed oil, sunflower oil, safflower oil, menhaden oil, salmon oil, herring oil, other vegetable or animal oils, oils of mineral origin or synthetic oils (including long chain fatty acid esters of glycerol or propylene glycol). In some instances, the oil naturally contains a high Z number element in sufficient quantity and can be used directly in the emulsion. In other instances, the oil is modified or derivatized to couple a high Z number element to the oil. Pharmaceutically acceptable oils are formulated by well known conventional methods (see: for example, Remington's Pharmaceutical Sciences, 18th Ed., Mack Publishing Co.).

Exemplary oils coupled to a high Z number atom of use in the emulsions of the invention are ethiodized oils which are organically combined iodine addition products of the ethyl ester of the fatty acid of poppy seed oil. Ethiodized oils, such as ethiodol and lipiodol, are non-ionic, iodinated radiopaque agents. Lipiodol is an iodinated derivative of poppy seed oil containing ethyl esters of linoleic, oleic, palmitic and stearic acids, with an iodine content of 38-40% w/v (see, for example, ABPI Data Sheet Compendium (1991-1992) The Pharmaceutical Industry, pp. 1199, Datapharm; London). Ethiodol is also a iodinated derivative of poppy seed oil but one in which iodine represents about 37% of the oil by weight.

Emulsifying agents, for example surfactants, are used to facilitate the formation of emulsions and increase their stability. Typically, aqueous phase surfactants have been used to facilitate the formation of oil-in-water emulsions. A surfactant is any substance that contains both hydrophilic and a hydrophobic portions. When added to water or solvents, a surfactant reduces the surface tension.

The lipid/surfactants used to form an outer coating on the nanoparticles (that can contain the coupled ligand or entrap reagents for binding desired components to the surface) include natural or synthetic phospholipids, fatty acids, cholesterols, lysolipids, sphingomyelins, tocopherols, glucolipids, stearylamines, cardiolipins, plasmalogens, a lipid with ether or ester linked fatty acids, and polymerized lipids. In some instances, the lipid/surfactant can include lipid conjugated polyethylene glycol (PEG). Various commercial anionic, cationic, and nonionic surfactants can also be employed, including Tweens, Spans, Tritons, and the like. In some embodiments, preferred surfactants are phospholipids and cholesterol.

Fluorinated surfactants which are soluble in the oil to be emulsified can also be used. Suitable fluorochemical surfactants include perfluorinated alkanoic acids such as perfluorohexanoic and perfluorooctanoic acids and amidoamine derivatives. These surfactants are generally used in amounts of 0.01 to 5.0% by weight, and preferably in amounts of 0.1 to 1.0%. Other suitable fluorochemical surfactants include perfluorinated alcohol phosphate esters and their salts; perfluorinated sulfonamide alcohol phosphate esters and their salts; perfluorinated alkyl sulfonamide; alkylene quaternary ammonium salts; N,N(carboxyl-substituted lower alkyl) perfluorinated alkyl sulfonamides; and mixtures thereof. As used herein, the term “perfluorinated” means that the surfactant contains at least one perfluorinated alkyl group.

Suitable perfluorinated alcohol phosphate esters include the free acids of the diethanolamine salts of mono- and bis(1H, 1H, 2H, 2H-perfluoroalkyl)phosphates. The phosphate salts, available under the tradename ZONYL RP (Dupont, Wilmington, Del.), are converted to the corresponding free acids by known methods. Suitable perfluorinated sulfonamide alcohol phosphate esters are described in U.S. Pat. No. 3,094,547. Suitable perfluorinated sulfonamide alcohol phosphate esters and salts of these include perfluoro-n-octyl-N-ethylsulfonamidoethyl phosphate, bis(perfluoro-n-octyl-N-ethylsulfonamidoethyl) phosphate, the ammonium salt of bis(perfluoro-n-octyl-N-ethylsulfonamidoethyl) phosphate,bis(perfluorodecyl-N-ethylsulfonamidoethyl)-phosphate and bis(perfluorohexyl-N ethylsulfonamidoethyl)phosphate. The preferred formulations use phosphatidylcholine, derivatized-phosphatidylethanolamine and cholesterol as the lipid surfactant.

Other known surfactant additives such as PLURONIC F-68, HAMPOSYL L30 (W.R. Grace Co., Nashua, N.H.), sodium dodecyl sulfate, Aerosol 413 (American Cyanamid Co., Wayne, N.J.), Aerosol 200 (American Cyanamid Co.), LIPOPROTEOL LCO (Rhodia Inc., Mammoth, N.J.), STANDAPOL SH 135 (Henkel Corp., Teaneck, N.J.), FIZUL 10-127 (Finetex Inc., Elmwood Park, N.J.), and CYCLOPOL SBFA 30 (Cyclo Chemicals Corp., Miami, Fla.); amphoterics, such as those sold with the trade names: Deriphat™ 170 (Henkel Corp.), LONZAINE JS (Lonza, Inc.), NIRNOL C2N-SF (Miranol Chemical Co., Inc., Dayton, N.J.), AMPHOTERGE W2 (Lonza, Inc.), and AMPHOTERGE 2WAS (Lonza, Inc.); non-ionics, such as those sold with the trade names: PLURONIC F-68 (BASF Wyandotte, Wyandotte, Mich.), PLURONIC F-127 (BASF Wyandotte), BRIJ 35 (ICI Americas; Wilmington, Del.), TRITON X-100 (Rohm and Haas Co., Philadelphia, Pa.), BRIJ 52 (ICI Americas), SPAN 20 (ICI Americas), GENEROL 122 ES (Henkel Corp.), TRITON N-42 (Rohm and Haas Co.), Triton™ N-101 (Rohm and Haas Co.), TRITON X-405 (Rohm and Haas Co.), TWEEN 80 (ICI Americas), TWEEN 85 (ICI Americas), and BRIJ 56 (ICI Americas) and the like, may be used alone or in combination in amounts of 0.10 to 5.0% by weight to assist in stabilizing the emulsions.

Lipid encapsulated emulsions may be formulated with cationic lipids in the surfactant layer that facilitate entrapping or adhering ligands, such as nucleic acids and aptamers, to particle surfaces. Typical cationic lipids may include DOTMA, N-[1-(2,3-dioleoyloxy)propyl]-N,N,N-trimethylammonium chloride; DOTAP, 1,2-dioleoyloxy-3-(trimethylammonio)propane; DOTB, 1,2-dioleoyl-3-(4′-trimethyl-ammonio)butanoyl-sn-glycerol, 1,2-diacyl-3-trimethylammonium-propane; DAP, 1,2-diacyl-3-dimethylammonium-propane; TAP, 1,2-diacyl-3-trimethylammonium-propane; 1,2-diacyl-sn-glycerol-3-ethyl phosphocholine; 3β-[N′,N′-dimethylaminoethane)-carbamol]cholesterol-HCl, DC-Cholesterol (DC-Chol); and DDAB, dimethyldioctadecylammonium bromide. In general the molar ratio of cationic lipid to non-cationic lipid in the lipid surfactant monolayer may be, for example, 1:1000 to 2:1, preferably, between 2:1 to 1:10, more preferably in the range between 1:1 to 1:2.5 and most preferably 1:1 (ratio of mole amount cationic lipid to mole amount non-cationic lipid, e.g., DPPC). A wide variety of lipids may comprise the non-cationic lipid component of the emulsion surfactant, particularly dipalmitoylphosphatidylcholine, dipalmitoylphosphatidyl-ethanolamine or dioleoylphosphatidylethanolamine in addition to those previously described. In lieu of cationic lipids as described above, lipids bearing cationic polymers such as polylysine or polyarginine may also be included in the lipid surfactant and afford binding of a negatively charged therapeutic, such as genetic material or analogues there of, to the outside of the emulsion particles. In some embodiments, the lipids can be cross-linked to provide stability to the emulsions for use in vivo. Emulsions with cross-linked lipids can be particularly useful for imaging methods described herein.

In particular embodiments, included in the lipid/surfactant coating are components with reactive groups that can be used to couple a targeting ligand and/or the ancillary substance useful for imaging or therapy. In some embodiments, a lipid/surfactant coating which provides a vehicle for binding a multiplicity of copies of one or more desired components to the nanoparticle is preferred. As will be described below, the lipid/surfactant components can be coupled to these reactive groups through functionalities contained in the lipid/surfactant component. For example, phosphatidylethanolamine may be coupled through its amino group directly to a desired moiety, or may be coupled to a linker such as a short peptide which may provide carboxyl, amino, or sulfhydryl groups as described below. Alternatively, standard linking agents such a maleimides may be used. A variety of methods may be used to associate the targeting ligand and the ancillary substances to the nanoparticles; these strategies may include the use of spacer groups such as polyethyleneglycol or peptides, for example.

The lipid/surfactant coated nanoparticles are typically formed by microfluidizing a mixture of the high Z number atom oil which forms the core and the lipid/surfactant mixture which forms the outer layer in suspension in aqueous medium to form an emulsion. In this procedure, the lipid/surfactants may already be coupled to additional ligands when they are emulsified into the nanoparticles, or may simply contain reactive groups for subsequent coupling. Alternatively, the components to be included in the lipid/surfactant layer may simply be solubilized in the layer by virtue of the solubility characteristics of the ancillary material. Sonication or other techniques may be required to obtain a suspension of the lipid/surfactant in the aqueous medium. Typically, at least one of the materials in the lipid/surfactant outer layer comprises a linker or functional group which is useful to bind the additional desired component or the component may already be coupled to the material at the time the emulsion is prepared.

For coupling by covalently binding the targeting ligand or other organic moiety (such as a chelating agent for a paramagnetic metal) to the components of the outer layer, various types of bonds and linking agents may be employed. Typical methods for forming such coupling include formation of amides with the use of carbodiamides, or formation of sulfide linkages through the use of unsaturated components such as maleimide. Other coupling agents include, for example, glutaraldehyde, propanedial or butanedial, 2-iminothiolane hydrochloride, bifunctional N-hydroxysuccinimide esters such as disuccinimidyl suberate, disuccinimidyl tartrate, bis[2-(succinimidooxycarbonyloxy)ethyl]sulfone, heterobifunctional reagents such as N-(5-azido-2-nitrobenzoyloxy)succinimide, succinimidyl 4-(N-maleimidomethyl)cyclohexane-1-carboxylate, and succinimidyl 4-(p-maleimidophenyl)butyrate, homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like. Linkage can also be accomplished by acylation, sulfonation, reductive amination, and the like. A multiplicity of ways to couple, covalently, a desired ligand to one or more components of the outer layer is well known in the art. The ligand itself may be included in the surfactant layer if its properties are suitable. For example, if the ligand contains a highly lipophilic portion, it may itself be embedded in the lipid/surfactant coating. Further, if the ligand is capable of direct adsorption to the coating, this too will effect its coupling. For example, nucleic acids, because of their negative charge, adsorb directly to cationic surfactants.

The ligand may bind directly to the nanoparticle, i.e., the ligand is associated with the nanoparticle itself. Alternatively, indirect binding may also be effected using a hydrolizable anchor, such as a hydrolizable lipid anchor, to couple the targeting ligand or other organic moiety to the lipid/surfactant coating of the emulsion. Indirect binding such as that effected through biotin/avidin may also be employed for the ligand. For example, in biotin/avidin mediated targeting, the targeting ligand is coupled not to the emulsion, but rather coupled, in biotinylated form to the targeted tissue.

Ancillary agents that may be coupled to the nanoparticles through entrapment in the coating layer include radionuclides. Radionuclides may be either therapeutic or diagnostic; diagnostic imaging using such nuclides is well known and by targeting radionuclides to desired tissue a therapeutic benefit may be realized as well. Radionuclides for diagnostic imaging often include gamma emitters (e.g., ⁹⁶Tc) and radionuclides for therapeutic purposes often include alpha emitters (e.g., ²²⁵Ac) and beta emitters (e.g., ⁹⁰Y). Typical diagnostic radionuclides include ^{99m}Tc, ⁹⁶Tc, ⁹⁵Tc, ¹¹¹In, ⁶²Cu, ⁶⁴Cu, ⁶⁷Ga, ⁶⁸Ga, ²⁰¹Tl, and ¹⁹²Ir, and therapeutic nuclides include ²²⁵Ac, ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, ¹³³Xe, ¹⁶⁹Yb, ¹⁷⁵Yb, ¹⁶⁵Dy, ¹⁶⁶Dy, ¹²³I, ¹³¹I, ⁶⁷Cu, ¹⁰⁵Rh, ¹¹¹Ag, and ¹⁹²Ir. The nuclide can be provided to a preformed emulsion in a variety of ways. For example, ⁹⁹Tc-pertechnate may be mixed with an excess of stannous chloride and incorporated into the preformed emulsion of nanoparticles. Stannous oxinate can be substituted for stannous chloride. In addition, commercially available kits, such as the HM-PAO (exametazine) kit marketed as Ceretek® by Nycomed Amersham can be used. Means to attach various radioligands to the nanoparticles of the invention are understood in the art.

Chelating agents containing metal ions for use in magnetic resonance imaging can also be employed as ancillary agents. Typically, a chelating agent containing a paramagnetic metal or superparamagnetic metal is associated with the lipids/surfactants of the coating on the nanoparticles and incorporated into the initial mixture which is sonicated. The chelating agent can be coupled directly to one or more of components of the coating layer. Suitable chelating agents are macrocyclic or linear chelating agents and include a variety of multi-dentate compounds including EDTA, DPTA, DOTA, and the like. These chelating agents can be coupled directly to functional groups contained in, for example, phosphatidyl ethanolamine, oleates, or any other synthetic natural or functionalized lipid or lipid soluble compound. Alternatively, these chelating agents can coupled through linking groups.

The paramagnetic and superparamagnetic metals useful in the MRI contrast agents of the invention include rare earth metals, typically, manganese, ytterbium, terbium, gadolinium, europium, and the like. Iron ions may also be used.

A particularly preferred set of MRI chelating agents includes 1,4,7,10-tetraazacyclododecane-1,4,7,10-tetraacetic acid (DOTA) and its derivatives, in particular, a methoxybenzyl derivative (MEO-DOTA) and a methoxybenzyl derivative comprising an isothiocyanate functional group (MEO-DOTA-NCS) which can then be coupled to the amino group of phosphatidyl ethanolamine or to a peptide derivatized form thereof. Derivatives of this type are described in U.S. Pat. No. 5,573,752 and other suitable chelating agents are disclosed in U.S. Pat. No. 6,056,939.

The DOTA isocyanate derivative can also be coupled to the lipid/surfactant directly or through a peptide spacer. The use of gly-gly-gly as a spacer is illustrated in the reaction scheme below. For direct coupling, the MEO-DOTA-NCS is simply reacted with phosphoethanolamine (PE) to obtain the coupled product. When a peptide is employed, for example a triglycyl link, PE is first coupled to t-boc protected triglycine. Standard coupling techniques, such as forming the activated ester of the free acid of the t-boc-triglycine using diisopropyl carbodiimide (or an equivalent thereof) with either N-hydroxy succinimide (NHS) or hydroxybenzotriazole (HBT) are employed and the t-boc-triglycine-PE is purified.

Treatment of the t-boc-triglycine-PE with trifluoroacetic acid yields triglycine-PE, which is then reacted with excess MEO-DOTA-NCS in DMF/CHCl₃ at 50° C. The final product is isolated by removing the solvent, followed by rinsing the remaining solid with excess water, to remove excess solvent and any un-reacted or hydrolyzed MEO-DOTA-NCS.

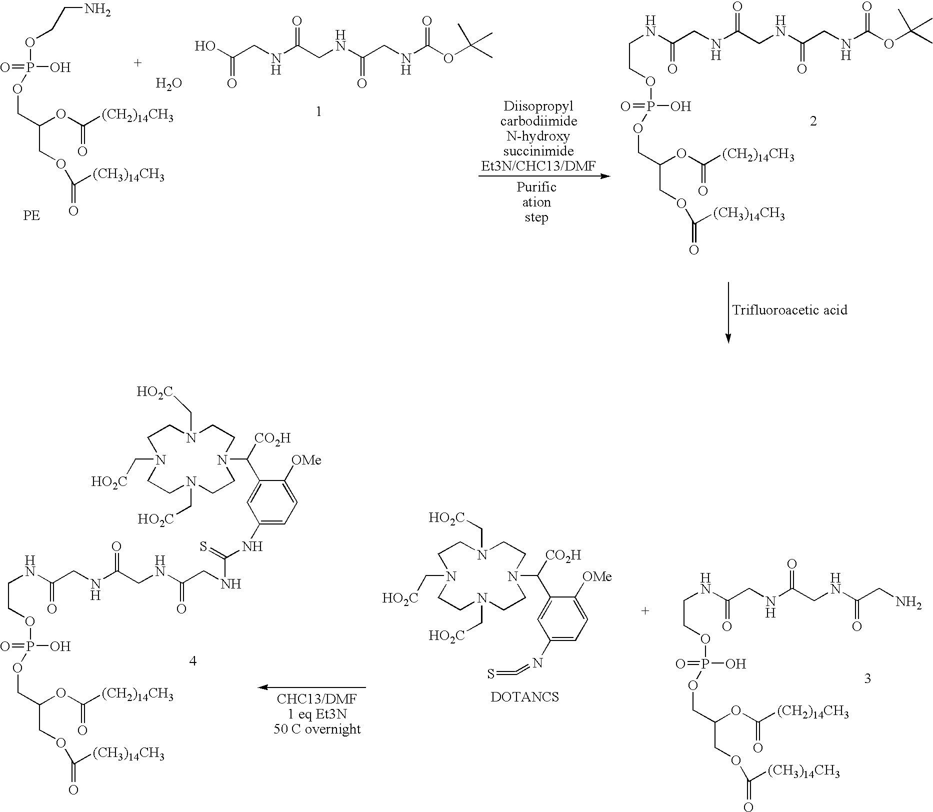

Other ancillary agents include fluorophores (such as fluorescein, dansyl, quantum dots, and the like) and infrared dyes or metals may be used in optical or light imaging (e.g., confocal microscopy and fluorescence imaging). For nuclear imaging, such as PET imaging, tosylated and ¹⁸F fluorinated compounds may be associated with the nanoparticles as ancillary agents.

In some embodiments, the biologically active agents are incorporated within the core of the emulsion nanoparticles with the oil coupled to a high Z number atom.

Included in the surface of the nanoparticle, in some embodiments of the invention, are biologically active agents. These biologically active agents can be of a wide variety, including proteins, nucleic acids, pharmaceuticals, and the like. Thus, included among suitable pharmaceuticals are antineoplastic agents, hormones, analgesics, anesthetics, neuromuscular blockers, antimicrobials or antiparasitic agents, antiviral agents, interferons, antidiabetics, antihistamines, antitussives, anticoagulants, and the like.

The targeted emulsions of the invention may also be used to provide a therapeutic agent combined with an imaging agent. Such emulsions would permit, for example, the site to be imaged in order to monitor the progress of the therapy on the site and to make desired adjustments in the dosage or therapeutic agent subsequently directed to the site. The invention thus provides a noninvasive means for the detection and therapeutic treatment of thrombi, infections, cancers and infarctions, for example, in patients while employing conventional imaging systems.

In all of the foregoing cases, whether the associated moiety is a targeting ligand or is an ancillary agent the defied moiety may be non-covalently associated with the lipid/surfactant layer, may be directly coupled to the components of the lipid/surfactant layer, or may be indirectly coupled to said components through spacer moieties.

As a specific example of a high Z number atom oil emulsion useful in the invention may be mentioned a ethiodol emulsion wherein the lipid coating thereof contains between approximately 50 to 99.5 mole percent lecithin, preferably approximately 55 to 70 to mole percent lecithin, 0 to 50 mole percent cholesterol, preferably approximately 25 to 45 mole percent cholesterol and approximately 0.5 to 10 mole percent biotinylated phosphatidylethanolamine, preferably approximately 1 to 5 mole percent biotinylated phosphatidylethanolamine. Other phospholipids such as phosphatidylserine may be biotinylated, fatty acyl groups such as stearylamine may be conjugated to biotin, or cholesterol or other fat soluble chemicals may be biotinylated and incorporated in the lipid coating for the lipid encapsulated particles. The preparation of an exemplary biotinylated high Z number atom oil emulsion for use in the practice of the invention is described hereinafter in accordance with known procedures.

The imaging and/or therapeutic target may be an in vivo or in vitro target and, preferably, a biological material although the target need not be a biological material. The target may be comprised of a surface to which the contrast substance binds or a three dimensional structure in which the contrast substance penetrates and binds to portions of the target below the surface.

Preferably, a ligand is incorporated into the contrast emulsion to immobilize or prolong the half-life of the emulsion nanoparticles at the imaging and/or therapeutic target. The ligand may be specific for a desired target to allow active targeting. Active targeting refers to ligand-directed, site-specific accumulation of agents to cells, tissues or organs by localization and binding to molecular epitopes, i.e., receptors, lipids, peptides, cell adhesion molecules, polysaccharides, biopolymers, and the like, presented on the surface membranes of cells or within the extracellular or intracellular matrix. A wide variety of ligands can be used including an antibody, a fragment of an antibody, a polypeptide such as small oligopeptide, a large polypeptide or a protein having three dimensional structure, a peptidomimetic, a polysaccharide, an aptamer, a lipid, a nucleic acid, a lectin or a combination thereof. Generally, the ligand specifically binds to a cellular epitope or receptor.

The term “ligand” as used herein is intended to refer to a targeting molecule that binds specifically to another molecule of a biological target separate and distinct from the emulsion particle itself. The reaction does not require nor exclude a molecule that donates or accepts a pair of electrons to form a coordinate covalent bond with a metal atom of a coordination complex. Thus a ligand may be attached covalently for direct-conjugation or noncovalently for indirect conjugation to the surface of the nanoparticle surface.

In some embodiments, for example for use in vivo, the binding affinity of the ligand for its specific target is about 10⁻⁷ M or greater. In some embodiments, for example, for use in vitro, the binding affinity of the ligand for its specific target can be less than 10⁻⁷ M.

Avidin-biotin interactions are extremely useful, noncovalent targeting systems that have been incorporated into many biological and analytical systems and selected in vivo applications. Avidin has a high affinity for biotin (10⁻¹⁵ M) facilitating rapid and stable binding under physiological conditions. Some targeted systems utilizing this approach are administered in two or three steps, depending on the formulation. Typically in these systems, a biotinylated ligand, such as a monoclonal antibody, is administered first and “pretargeted” to the unique molecular epitopes. Next, avidin is administered, which binds to the biotin moiety of the “pretargeted” ligand. Finally, the biotinylated emulsion is added and binds to the unoccupied biotin-binding sites remaining on the avidin thereby completing the ligand-avidin-emulsion “sandwich.” The avidin-biotin approach can avoid accelerated, premature clearance of targeted agents by the reticuloendothelial system secondary to the presence of surface antibody. Additionally, avidin, with four, independent biotin binding sites provides signal amplification and improves detection sensitivity.

As used herein, the term “biotin emulsion” or “biotinylated” with respect to conjugation to a biotin emulsion or biotin agent is intended to include biotin, biocytin and other biotin derivatives and analogs such as biotin amido caproate N-hydroxysuccinimide ester, biotin 4-amidobenzoic acid, biotinamide caproyl hydrazide and other biotin derivatives and conjugates. Other derivatives include biotin-dextran, biotin-disulfide N-hydroxysuccinimide ester, biotin-6 amido quinoline, biotin hydrazide, d-biotin-N hydroxysuccinimide ester, biotin maleimide, d-biotin p-nitrophenyl ester, biotinylated nucleotides and biotinylated amino acids such as N, epsilon-biotinyl-l-lysine. The term “avidin emulsion” or “avidinized” with respect to conjugation to an avidin emulsion or avidin agent is intended to include avidin, streptavidin and other avidin analogs such as streptavidin or avidin conjugates, highly purified and fractionated species of avidin or streptavidin, and non-amino acid or partial-amino acid variants, recombinant or chemically synthesized avidin.

Targeting ligands may be chemically attached to the surface of nanoparticles of the emulsion by a variety of methods depending upon the nature of the particle surface. Conjugations may be performed before or after the emulsion particle is created depending upon the ligand employed. Direct chemical conjugation of ligands to proteinaceous agents often take advantage of numerous amino-groups (e.g. lysine) inherently present within the surface. Alternatively, functionally active chemical groups such as pyridyldithiopropionate, maleimide or aldehyde may be incorporated into the surface as chemical “hooks” for ligand conjugation after the particles are formed. Another common post-processing approach is to activate surface carboxylates with carbodiimide prior to ligand addition. The selected covalent linking strategy is primarily determined by the chemical nature of the ligand. Antibodies and other large proteins may denature under harsh processing conditions; whereas, the bioactivity of carbohydrates, short peptides, aptamers, drugs or peptidomimetics often can be preserved. To ensure high ligand binding integrity and maximize targeted particle avidity flexible polymer spacer arms, e.g. polyethylene glycol or simple caproate bridges, can be inserted between an activated surface functional group and the targeting ligand. These extensions can be 10 nm or longer and minimize interference of ligand binding by particle surface interactions.

Antibodies, particularly monoclonal antibodies, may also be used as site-targeting ligands directed to any of a wide spectrum of molecular epitopes including pathologic molecular epitopes. Immunoglobin-γ (IgG) class monoclonal antibodies have been conjugated to liposomes, emulsions and other microbubble particles to provide active, site-specific targeting. Generally, these proteins are symmetric glycoproteins (MW ca. 150,000 Daltons) composed of identical pairs of heavy and light chains. Hypervariable regions at the end of each of two arms provide identical antigen-binding domains. A variably sized branched carbohydrate domain is attached to complement-activating regions, and the hinge area contains particularly accessible interchain disulfide bonds that may be reduced to produce smaller fragments.

Preferably, monoclonal antibodies are used in the antibody compositions of the invention. Monoclonal antibodies specific for selected antigens on the surface of cells may be readily generated using conventional techniques (see, for example, U.S. Pat. Nos. RE 32,011, 4,902,614, 4,543,439, and 4,411,993). Hybridoma cells can be screened immunochemically for production of antibodies specifically reactive with an antigen, and monoclonal antibodies can be isolated. Other techniques may also be utilized to construct monoclonal antibodies (see, for example, Huse et al. (1989) Science 246:1275-1281; Sastry et al. (1989) Proc. Natl. Acad. Sci. USA 86:5728-5732; Alting-Mees et al. (1990) Strategies in Molecular Biology 3:1-9).

Within the context of the present invention, antibodies are understood to include various kinds of antibodies, including, but not necessarily limited to, naturally occurring antibodies, monoclonal antibodies, polyclonal antibodies, antibody fragments that retain antigen binding specificity (e.g., Fab, and F(ab′)₂) and recombinantly produced binding partners, single domain antibodies, hybrid antibodies, chimeric antibodies, single-chain antibodies, human antibodies, humanized antibodies, and the like. Generally, antibodies are understood to be reactive against a selected antigen of a cell if they bind with an affinity (association constant) of greater than or equal to 10⁷ M⁻¹. Antibodies against selected antigens for use with the emulsions may be obtained from commercial sources.

Further description of the various kinds of antibodies of use as site-targeting ligands in the invention is provided herein, in particular, later in this Compositions of the Invention section.

The emulsions of the present invention also employ targeting agents that are ligands other than an antibody or fragment thereof. For example, polypeptides, like antibodies, may have high specificity and epitope affinity for use as vector molecules for targeted contrast agents. These may be small oligopeptides, having, for example, 5 to 10 amino acid, specific for a unique receptor sequences (such as, for example, the RGD epitope of the platelet GIIbIIIa receptor) or larger, biologically active hormones such as cholecystokinin. Smaller peptides potentially have less inherent immunogenicity than nonhumanized murine antibodies. Peptides or peptide (nonpeptide) analogues of cell adhesion molecules, cytokines, selectins, cadhedrins, Ig superfamily, integrins and the like may be utilized for targeted imaging and/or therapeutic delivery.

In some instances, the ligand is a non-peptide organic molecule, such as those described in U.S. Pat. Nos. 6,130,231 (for example as set forth in formula 1); 6,153,628; 6,322,770; and PCT publication WO 01/97848. “Non-peptide” moieties in general are those other than compounds which are simply polymers of amino acids, either gene encoded or non-gene encoded. Thus, “non-peptide ligands” are moieties which are commonly referred to as “small molecules” lacking in polymeric character and characterized by the requirement for a core structure other than a polymer of amino acids. The non-peptide ligands useful in the invention may be coupled to peptides or may include peptides coupled to portions of the ligand which are responsible for affinity to the target site, but it is the non-peptide regions of this ligand which account for its binding ability. For example, non-peptide ligands specific for the αᵥβ₃ integrin are described in U.S. Pat. Nos. 6,130,231 and 6,153,628.

Carbohydrate-bearing lipids may be used for targeting of the emulsions, as described, for example, in U.S. Pat. No. 4,310,505.

Asialoglycoproteins have been used for liver-specific applications due to their high affinity for asialoglycoproteins receptors located uniquely on hepatocytes. Asialoglycoproteins directed agents (primarily magnetic resonance agents conjugated to iron oxides) have been used to detect primary and secondary hepatic tumors as well as benign, diffuse liver disease such as hepatitis. The asialoglycoproteins receptor is highly abundant on hepatocytes, approximately 500,000 per cell, rapidly internalizes and is subsequently recycled to the cell surface. Polysaccharides such as arabinogalactan may also be utilized to localize emulsions to hepatic targets. Arabinogalactan has multiple terminal arabinose groups that display high affinity for asialoglycoproteins hepatic receptors.

Aptamers are high affinity, high specificity RNA or DNA-based ligands produced by in vitro selection experiments (SELEX: systematic evolution of ligands by exponential enrichment). Aptamers are generated from random sequences of 20 to 30 nucleotides, selectively screened by absorption to molecular antigens or cells, and enriched to purify specific high affinity binding ligands. To enhance in vivo stability and utility, aptamers are generally chemically modified to impair nuclease digestion and to facilitate conjugation with drugs, labels or particles. Other, simpler chemical bridges often substitute nucleic acids not specifically involved in the ligand interaction. In solution aptamers are unstructured but can fold and enwrap target epitopes providing specific recognition. The unique folding of the nucleic acids around the epitope affords discriminatory intermolecular contacts through hydrogen bonding, electrostatic interaction, stacking, and shape complementarity. In comparison with protein-based ligands, generally aptamers are stable, are more conducive to heat sterilization, and have lower immunogenicity. Aptamers are currently used to target a number of clinically relevant pathologies including angiogenesis, activated platelets, and solid tumors and their use is increasing. The clinical effectiveness of aptamers as targeting ligands for imaging and/or therapeutic emulsion particles may be dependent upon the impact of the negative surface charge imparted by nucleic acid phosphate groups on clearance rates. Previous research with lipid-based particles suggest that negative zeta potentials markedly decrease liposome circulatory half-life, whereas, neutral or cationic particles have similar, longer systemic persistence.

It is also possible to use what has been referred to as a “primer material” to couple specific binding species to the emulsion for certain applications. As used herein, “primer material” refers to any constituent or derivatized constituent incorporated into the emulsion lipid surfactant layer that could be chemically utilized to form a covalent bond between the particle and a targeting ligand or a component of the targeting ligand such as a subunit thereof.

Thus, the specific binding species (i.e. targeting ligand) may be immobilized on the encapsulating lipid monolayer by direct adsorption to the oil/aqueous interface or using a primer material. A primer material may be any surfactant compatible compound incorporated in the particle to chemically couple with or adsorb a specific binding or targeting species. The preferred result is achieved by forming an emulsion with an aqueous continuous phase and a biologically active ligand adsorbed or conjugated to the primer material at the interface of the continuous and discontinuous phases. Naturally occurring or synthetic polymers with amine, carboxyl, mercapto, or other functional groups capable of specific reaction with coupling agents and highly charged polymers may be utilized in the coupling process. The specific binding species (e.g. antibody) may be immobilized on the oil coupled to a high Z number atom emulsion particle surface by direct adsorption or by chemical coupling. Examples of specific binding species which can be immobilized by direct adsorption include small peptides, peptidomimetics, or polysaccharide-based agents. To make such an emulsion the specific binding species may be suspended or dissolved in the aqueous phase prior to formation of the emulsion. Alternatively, the specific binding species may be added after formation of the emulsion and incubated with gentle agitation at room temperature (about 25° C.) in a pH 7.0 buffer (typically phosphate buffered saline) for 1.2 to 18 hours.

Where the specific binding species is to be coupled to a primer material, conventional coupling techniques may be used. The specific binding species may be covalently bonded to primer material with coupling agents using methods which are known in the art. Primer materials may include phosphatidylethanolamine (PE), N-caproylamine-PE, n-dodecanylamine, phosphatidylthioethanol,N-1,2-diacyl-sn-glycero-3-phosphoethanolamine-N-[4-(p-maleimidophenyl)butyramide], 1,2-diacyl-sn-glycero-3-phosphoethanolamine-N-[4-(p-maleimidomethyl)cyclohexane-carboxylate], 1,2-diacyl-sn-glycero-3-phosphoethanolamine-N-[3-(2-pyridyldithio)propionate], 1,2-diacyl-sn-glycero-3-phosphoethanolamine-N[PDP(polyethylene glycol)2000], N-succinyl-PE, N-glutaryl-PE, N-dodecanyl-PE, N-biotinyl-PE, or N-caproyl-PE. Additional coupling agents include, for example, use a carbodiimide or an aldehyde having either ethylenic unsaturation or having a plurality of aldehyde groups. Further description of additional coupling agents appropriate for use is provided herein, in particular, later in this Compositions of the Invention section.

Covalent bonding of a specific binding species to the primer material can be carried out with the reagents provided herein by conventional, well-known reactions, for example, in the aqueous solutions at a neutral pH, at temperatures of less than 25° C. for 1 hour to overnight. Examples of linkers for coupling a ligand, including non-peptide ligands, are known in the art.

Emulsifying and/or solubilizing agents may also be used in conjunction with emulsions. Such agents include, but are not limited to, acacia, cholesterol, diethanolamine, glyceryl monostearate, lanolin alcohols, lecithin, mono- and di-glycerides, mono-ethanolamine, oleic acid, oleyl alcohol, poloxamer, peanut oil, palmitic acid, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax. All lipids with perfluoro fatty acids as a component of the lipid in lieu of the saturated or unsaturated hydrocarbon fatty acids found in lipids of plant or animal origin may be used. Suspending and/or viscosity-increasing agents that may be used with emulsions include, but are not limited to, acacia, agar, alginic acid, aluminum mono-stearate, bentonite, magma, carbomer 934P, carboxymethylcellulose, calcium and sodium and sodium 12, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, povidone, propylene glycol alginate, silicon dioxide, sodium alginate, tragacanth, and xanthum gum.

As described herein, emulsions of the invention may incorporate bioactive agents (e.g. drugs, prodrugs, genetic materials, radioactive isotopes, or combinations thereof) in their native form or derivatized with hydrophobic or charged moieties to enhance incorporation or adsorption to the nanoparticle. In particular, bioactive agents may be incorporated in targeted emulsions of the invention. The bioactive agent may be a prodrug, including the prodrugs described, for example, by Sinkyla et al. (1975) J. Pharm. Sci. 64:181-210, Koning et al. (1999) Br. J. Cancer 80:1718-1725, U.S. Pat. No. 6,090,800 and U.S. Pat. No. 6,028,066.

Such therapeutic emulsions may also include, but are not limited to antineoplastic agents, radiopharmaceuticals, protein and nonprotein natural products or analogues/mimetics thereof including hormones, analgesics, muscle relaxants, narcotic agonists, narcotic agonist-antagonists, narcotic antagonists, nonsteroidal anti-inflammatories, anesthetic and sedatives, neuromuscular blockers, antimicrobials, anti-helmintics, antimalarials, antiparasitic agents, antiviral agents, antiherpetic agents, antihypertensives, antidiabetic agents, gout related medicants, antihistamines, antiulcer medicants, anticoagulants and blood products.

Genetic material, includes, for example, nucleic acids, RNA and DNA, of either natural or synthetic origin, including recombinant RNA and DNA and antisense RNA and DNA; hammerhead RNA, ribozymes, hammerhead ribozymes, antigene nucleic acids, both single and double stranded RNA and DNA and analogs thereof, immunostimulatory nucleic acid, ribooligonucleotides, antisense ribooligonucleotides, deoxyribooligonucleotides, and antisense deoxyribooligonucleotides. Other types of genetic material that may be used include, for example, genes carried on expression vectors such as plasmids, phagemids, cosmids, yeast artificial chromosomes, and defective or “helper” viruses, antigene nucleic acids, both single and double stranded RNA and DNA and analogs thereof, such as phosphorothioate and phosphorodithioate oligodeoxynucleotides. Additionally, the genetic material may be combined, for example, with proteins or other polymers.

Further description of additional therapeutic agents appropriate for use is provided herein, in particular, later in this Compositions of the Invention section.

As described herein, the emulsion nanoparticles may incorporate on the particle paramagnetic or super paramagnetic elements including but not limited to gadolinium, magnesium, iron, manganese in their native or in a chemically complexed form. Similarly, radioactive nuclides including positron-emitters, gamma-emitters, beta-emitters, alpha-emitters in their native or chemically-complexed form may be included on or in the particles. Adding of these moieties permits the additional use of other clinical imaging modalities such as magnetic resonance imaging, positron emission tomography, and nuclear medicine imaging techniques in conjunction with X-ray and ultrasonic imaging.

In addition, optical imaging, which refers to the production of visible representations of tissue or regions of a patient produced by irradiating those tissues or regions of a patient with electromagnetic energy in the spectral range between ultraviolet and infrared, and analyzing either the reflected, scattered, absorbed and/or fluorescent energy produced as a result of the irradiation, may be combined with the X-ray imaging of targeted emulsions. Examples of optical imaging include, but are not limited to, visible photography and variations thereof, ultraviolet images, infrared images, fluorimetry, holography, visible microscopy, fluorescent microscopy, spectrophotometry, spectroscopy, fluorescence polarization and the like.

Photoactive agents, i.e. compounds or materials that are active in light or that responds to light, including, for example, chromophores (e.g., materials that absorb light at a given wavelength), fluorophores (e.g., materials that emit light at a given wavelength), photosensitizers (e.g., materials that can cause necrosis of tissue and/or cell death in vitro and/or in vivo), fluorescent materials, phosphorescent materials and the like, that may be used in diagnostic or therapeutic applications. “Light” refers to all sources of light including the ultraviolet (UV) region, the visible region and/or the infrared (IR) region of the spectrum. Suitable photoactive agents that may be used in the present invention have been described by others (for example, U.S. Pat. No. 6,123,923). Further description of additional photoactive agents appropriate for use is provided herein, in particular, later in this Compositions of the Invention section.

In addition, certain ligands, such as, for example, antibodies, peptide fragments, or mimetics of a biologically active ligand may contribute to the inherent therapeutic effects, either as an antagonistic or agonistic, when bound to specific epitopes. As an example, antibody against αᵥβ₃ integrin on neovascular endothelial cells has been shown to transiently inhibit growth and metastasis of solid tumors. The efficacy of therapeutic emulsion particles directed to the αᵥβ₃ integrin may result from the improved antagonistic action of the targeting ligand in addition to the effect of the therapeutic agents incorporated and delivered by particle itself.

Useful emulsions may have a wide range of nominal particle diameters, e.g., from as small as about 0.01 μm to as large as 10 μm, preferably about 50 nm to about 1000 nm, more preferably about 50 nm to about 500 nm, in some instances about 50 nm to about 300 nm, in some instances about 100 nm to about 300 nm, in some instances about 200 nm to about 250 nm, in some instances about 200 nm, in some instances about less than 200 nm. Generally, small size particles, for example, submicron particles, circulate longer and tend to be more stable than larger particles.

In addition to that described elsewhere herein, following is further description of the various kinds of antibodies appropriate for use as site-targeting ligands in and/or with the emulsions of the invention.

Bivalent F(ab′)₂ and monovalent F(ab) fragments can be used as ligands and these are derived from selective cleavage of the whole antibody by pepsin or papain digestion, respectively. Antibodies can be fragmented using conventional techniques and the fragments (including “Fab” fragments) screened for utility in the same manner as described above for whole antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab)₂), which are capable of selectively reacting with a designated antigen or antigen family. Methods of producing Fab fragments of antibodies are known within the art and include, for example, proteolysis, and synthesis by recombinant techniques. For example, F(ab′)₂ fragments can be generated by treating antibody with pepsin. The resulting F(ab′)₂ fragment can be treated to reduce disulfide bridges to produce Fab′ fragments. “Fab” antibodies may be divided into subsets analogous to those described herein, i.e., “hybrid Fab”, “chimeric Fab”, and “altered Fab”. Elimination of the Fc region greatly diminishes the immunogenicity of the molecule, diminishes nonspecific liver uptake secondary to bound carbohydrate, and reduces complement activation and resultant antibody-dependent cellular toxicity. Complement fixation and associated cellular cytotoxicity can be detrimental when the targeted site must be preserved or beneficial when recruitment of host killer cells and target-cell destruction is desired (e.g. anti-tumor agents).

Most monoclonal antibodies are of murine origin and are inherently immunogenic to varying extents in other species. Humanization of murine antibodies through genetic engineering has lead to development of chimeric ligands with improved biocompatibility and longer circulatory half-lives. Antibodies used in the invention include those that have been humanized or made more compatible with the individual to which they will be administered. In some cases, the binding affinity of recombinant antibodies to targeted molecular epitopes can be improved with selective site-directed mutagenesis of the binding idiotype. Methods and techniques for such genetic engineering of antibody molecules are known in the art. By “humanized” is meant alteration of the amino acid sequence of an antibody so that fewer antibodies and/or immune responses are elicited against the humanized antibody when it is administered to a human. For the use of the antibody in a mammal other than a human, an antibody may be converted to that species format.

Phage display techniques may be used to produce recombinant human monoclonal antibody fragments against a large range of different antigens without involving antibody-producing animals. In general, cloning creates large genetic libraries of corresponding DNA (cDNA) chains deducted and synthesized by means of the enzyme “reverse transcriptase” from total messenger RNA (mRNA) of human B lymphocytes. By way of example, immunoglobulin cDNA chains are amplified by polymerase chain reaction (PCR) and light and heavy chains specific for a given antigen are introduced into a phagemid vector. Transfection of this phagemid vector into the appropriate bacteria results in the expression of an scFv immunoglobulin molecule on the surface of the bacteriophage. Bacteriophages expressing specific immunoglobulin are selected by repeated immunoadsorption/phage multiplication cycles against desired antigens (e.g., proteins, peptides, nuclear acids, and sugars). Bacteriophages strictly specific to the target antigen are introduced into an appropriate vector, (e.g., Escherichia coli, yeast, cells) and amplified by fermentation to produce large amounts of human antibody fragments, generally with structures very similar to natural antibodies. Phage display techniques are known in the art and have permitted the production of unique ligands for targeting and therapeutic applications.

Polyclonal antibodies against selected antigens may be readily generated by one of ordinary skill in the art from a variety of warm-blooded animals such as horses, cows, various fowl, rabbits, mice, or rats. In some cases, human polyclonal antibodies against selected antigens may be purified from human sources.

As used herein, a “single domain antibody” (dAb) is an antibody which is comprised of a V_{H} domain, which reacts immunologically with a designated antigen. A dAb does not contain a V_{L} domain, but may contain other antigen binding domains known to exist in antibodies, for example, the kappa and lambda domains. Methods for preparing dAbs are known in the art. See, for example, Ward et al. (1989) Nature 341:544-546. Antibodies may also be comprised of V_{H} and V_{L} domains, as well as other known antigen binding domains. Examples of these types of antibodies and methods for their preparation are known in the art (see, e.g., U.S. Pat. No. 4,816,467).

Further exemplary antibodies include “univalent antibodies”, which are aggregates comprised of a heavy chain/light chain dimer bound to the Fc (i.e., constant) region of a second heavy chain. This type of antibody generally escapes antigenic modulation. See, e.g., Glennie et al. (1982) Nature 295:712-714.

“Hybrid antibodies” are antibodies wherein one pair of heavy and light chains is homologous to those in a first antibody, while the other pair of heavy and light chains is homologous to those in a different second antibody. Typically, each of these two pairs will bind different epitopes, particularly on different antigens. This results in the property of “divalence”, i.e., the ability to bind two antigens simultaneously. Such hybrids may also be formed using chimeric chains, as set forth herein.

The invention also encompasses “altered antibodies”, which refers to antibodies in which the naturally occurring amino acid sequence in a vertebrate antibody has been varied. Utilizing recombinant DNA techniques, antibodies can be redesigned to obtain desired characteristics. The possible variations are many, and range from the changing of one or more amino acids to the complete redesign of a region, for example, the constant region. Changes in the variable region may be made to alter antigen binding characteristics. The antibody may also be engineered to aid the specific delivery of an emulsion to a specific cell or tissue site. The desired alterations may be made by known techniques in molecular biology, e.g., recombinant techniques, site directed mutagenesis, and other techniques.

“Chimeric antibodies”, are antibodies in which the heavy and/or light chains are fusion proteins. Typically the constant domain of the chains is from one particular species and/or class, and the variable domains are from a different species and/or class. The invention includes chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of targeted cells and/or tissues. See, for example, Morrison et al. (1985) Proc. Natl. Acad. Sci. U.S.A. 81:6851; Takeda et al. (1985) Nature 314:452; U.S. Pat. Nos. 4,816,567 and 4,816,397; European Patent Publications EP171496 and EP173494; United Kingdom patent GB 2177096B.

Bispecific antibodies may contain a variable region of an anti-target site antibody and a variable region specific for at least one antigen on the surface of the lipid-encapsulated emulsion. In other cases, bispecific antibodies may contain a variable region of an anti-target site antibody and a variable region specific for a linker molecule. Bispecific antibodies may be obtained forming hybrid hybridomas, for example by somatic hybridization. Hybrid hybridomas may be prepared using the procedures known in the art such as those disclosed in Staerz et al. (1986, Proc. Natl. Acad. Sci. U.S.A. 83:1453) and Staerz et al. (1986, Immunology Today 7:241). Somatic hybridization includes fusion of two established hybridomas generating a quadroma (Milstein et al. (1983) Nature 305:537-540) or fusion of one established hybridoma with lymphocytes derived from a mouse immunized with a second antigen generating a trioma (Nolan et al. (1990) Biochem. Biophys. Acta 1040:1-11). Hybrid hybridomas are selected by making each hybridoma cell line resistant to a specific drug-resistant marker (De Lau et al. (1989) J. Immunol. Methods 117:1-8), or by labeling each hybridoma with a different fluorochrome and sorting out the heterofluorescent cells (Karawajew et al. (1987) J. Immunol. Methods 96:265-270).

Bispecific antibodies may also be constructed by chemical means using procedures such as those described by Staerz et al. (1985) Nature 314:628 and Perez et al. (1985) Nature 316:354. Chemical conjugation may be based, for example, on the use of homo- and heterobifunctional reagents with E-amino groups or hinge region thiol groups. Homobifunctional reagents such as 5,5′-dithiobis(2-nitrobenzoic acid) (DNTB) generate disulfide bonds between the two Fabs, and O-phenylenedimaleimide (O-PDM) generate thioether bonds between the two Fabs (Brenner et al. (1985) Cell 40:183-190, Glennie et al. (1987) J. Immunol. 139:2367-2375). Heterobifunctional reagents such as N-succinimidyl-3-(2-pyridylditio) propionate (SPDP) combine exposed amino groups of antibodies and Fab fragments, regardless of class or isotype (Van Dijk et al. (1989) Int. J. Cancer 44:738-743).

Bifunctional antibodies may also be prepared by genetic engineering techniques. Genetic engineering involves the use of recombinant DNA based technology to ligate sequences of DNA encoding specific fragments of antibodies into plasmids, and expressing the recombinant protein. Bispecific antibodies can also be made as a single covalent structure by combining two single chains Fv (scFv) fragments using linkers (Winter et al. (1991) Nature 349:293-299); as leucine zippers coexpressing sequences derived from the transcription factors fos and jun (Kostelny et al. (1992) J. Immunol. 148:1547-1553); as helix-turn-helix coexpressing an interaction domain of p53 (Rheinnecker et al. (1996) J. Immunol. 157:2989-2997), or as diabodies (Holliger et al. (1993) Proc. Natl. Acad. Sci. U.S.A. 90:6444-6448).

In addition to that described elsewhere herein, following is further description of coupling agents appropriate for use in coupling a primer material, for example, to a specific binding or targeting ligand. Additional coupling agents use a carbodiimide such as 1-ethyl-3-(3-N,N dimethylaminopropyl) carbodiimide hydrochloride or 1-cyclohexyl-3-(2-morpholinoethyl)carbodiimide methyl-p-toluenesulfonate. Other suitable coupling agents include aldehyde coupling agents having either ethylenic unsaturation such as acrolein, methacrolein, or 2-butenal, or having a plurality of aldehyde groups such as glutaraldehyde, propanedial or butanedial. Other coupling agents include 2-iminothiolane hydrochloride, bifunctional N-hydroxysuccinimide esters such as disuccinimidyl substrate, disuccinimidyl tartrate, bis[2-(succinimidooxycarbonyloxy)ethyl]sulfone, disuccinimidyl propionate, ethylene glycolbis(succinimidyl succinate); heterobifunctional reagents such as N-(5-azido-2-nitrobenzoyloxy)succinimide, p-azidophenylbromide, p-azidophenylglyoxal, 4-fluoro-3-nitrophenylazide, N-hydroxysuccinimidyl-4-azidobenzoate, m-maleimidobenzoyl N-hydroxysuccinimide ester, methyl-4-azidophenylglyoxal, 4-fluoro-3-nitrophenyl azide, N-hydroxysuccinimidyl-4-azidobenzoate hydrochloride, p-nitrophenyl 2-diazo-3,3,3-trifluoropropionate, N-succinimidyl-6-(4′-azido-2′-nitrophenylamino)hexanoate, succinimidyl 4-(N-maleimidomethyl)cyclohexane-1-carboxylate, succinimidyl 4-(p-maleimidophenyl)butyrate, N-succinimidyl(4-azidophenyldithio)propionate, N-succinmidyl 3-(2-pyridyldithio)propionate, N-(4-azidophenylthio)phthalamide; homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like.

In addition to that described elsewhere herein, following is further description of therapeutic agents that may be incorporated onto and/or within the nanoparticles of the invention. Generally, the therapeutic agents can be derivatized with a lipid anchor to make the agent lipid soluble or to increase its solubility in lipid, therefor increasing retension of the agent in the lipid layer of the emulsion and/or in the lipid membrane of the target cell. Such therapeutic emulsions may also include, but are not limited to antineoplastic agents, including platinum compounds (e.g., spiroplatin, cisplatin, and carboplatin), methotrexate, fluorouracil, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, taxol, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, interferon α-2a, interferon α-2b, teniposide (VM-26), vinblastine sulfate (VLB), vincristine sulfate, bleomycin, bleomycin sulfate, methotrexate, adriamycin, arabinosyl, hydroxyurea, procarbazine, dacarbazine, mitotic inhibitors such as etoposide and other vinca alkaloids; radiopharmaceuticals such as but not limited to radioactive iodine, samarium, strontium cobalt, yittrium and the like; protein and nonprotein natural products or analogues/mimetics thereof including hormones such as but not limited to growth hormone, somatostatin, prolactin, thyroid, steroids, androgens, progestins, estrogens and antiestrogens; analgesics including but not limited to antirheumatics, such as auranofin, methotrexate, azathioprine, sulfazalazine, leflunomide, hydrochloroquine, and etanercept; muscle relaxants such as baclofen, dantrolene, carisoprodol, diazepam, metaxalone, cyclobenzaprine, chlorzoxazone, tizanidine; narcotic agonists such as codeine, fentanyl, hydromorphone, lleavorphanol, meperidine, methadone, morphine, oxycodone, oxymorphone, propoxyphene; narcotic agonist-antagonists such as buprenorphine, butorphanol, dezocine, nalbuphine, pentazocine; narcotic antagonists such as nalmefene and naloxone, other analgesics including ASA, acetominophen, tramadol, or combinations thereof; nonsteroidal anti-inflammatories including but not limited to celecoxib, diclofenac, diflunisal, etodolac, fenoprofen, flurbiprofen, ibuprofen, indomethacin, ketoprofen, ketorolac, naproxen, oxaproxen, rofecoxib, salisalate, suldindac, tolmetin; anesthetic and sedatives such as etomidate, fentanyl, ketamine, methohexital, propofol, sufentanil, thiopental, and the like; neuromuscular blockers such as but not limited to pancuronium, atracurium, cisatracurium, rocuronium, succinylcholine, vercuronium; antimicrobials including aminoglycosides, antifungal agents including amphotericin B, clotrimazole, fluconazole, flucytosine, griseofulvin, itraconazole, ketoconazole, nystatin, and terbinafine; anti-helmintics; antimalarials, such as chloroquine, doxycycline, mefloquine, primaquine, quinine; antimycobacterial including dapsone, ethambutol, ethionamide, isoniazid, pyrazinamide, rifabutin, rifampin, rifapentine; antiparasitic agents including albendazole, atovaquone, iodoquinol, ivermectin, mebendazole, metronidazole, pentamidine, praziquantel, pyrantel, pyrimethamine, thiabendazole; antiviral agents including abacavir, didanosine, lamivudine, stavudine, zalcitabine, zidovudine as well as protease inhibitors such as indinavir and related compounds, anti-CMV agents including but not limited to cidofovir, foscamet, and ganciclovir; antiherpetic agents including amatadine, rimantadine, zanamivir; interferons, ribavirin, rebetron; carbapenems, cephalosporins, fluoroquinones, macrolides, penicillins, sulfonamides, tetracyclines, and other antimicrobials including aztreonam, chloramphenieol, fosfomycin, furazolidone, nalidixic acid, nitrofurantoin, vancomycin and the like; nitrates, antihypertensives including diuretics, beta blockers, calcium channel blockers, angiotensin converting enzyme inhibitors, angiotensin receptor antagonists, antiadrenergic agents, anti-dysrhythmics, antihyperlipidemic agents, antiplatelet compounds, pressors, thrombolytics, acne preparations, antipsoriatics; corticosteroids; androgens, anabolic steroids, bisphosphonates; sulfonoureas and other antidiabetic agents; gout related medicants; antihistamines, antitussive, decongestants, and expectorants; antiulcer medicants including antacids, 5-HT receptor antagonists, H2-antagonists, bismuth compounds, proton pump inhibitors, laxatives, octreotide and its analogues/mimetics; anticoagulants; immunization antigens, immunoglobins, immunosuppressive agents; anticonvulsants, 5-HT receptor agonists, other migraine therapies; parkinsonian agents including anticholinergics, and dopaminergics; estrogens, GnRH agonists, progestins, estrogen receptor modulators, tocolytics, uterotnics, thyroid agents such as iodine products and anti-thyroid agents; blood products such as parenteral iron, hemin, hematoporphyrins and their derivatives.

In addition to that described elsewhere herein, following is further description of additional photoactive agents appropriate for use in optical imaging of the nanoparticles of the invention. Suitable photoactive agents include but are not limited to, for example, fluoresceins, indocyanine green, rhodamine, triphenylmethines, polymethines, cyanines, fullerenes, oxatellurazoles, verdins, rhodins, perphycenes, sapphyrins, rubyrins, cholesteryl 4,4-difluoro-5,7-dimethyl-4-bora-3a,4a-diaza-s-indacene-3-dodecanoate, cholesteryl 12-(N-methyl-N-(7-nitrobenz-2-oxa-1,3-diazol-4-yl)amino)dodecanate, cholesteryl cis-parinarate, cholesteryl 3-((6-phenyl)-1,3,5-hexatrienyl)phenyl-proprionate, cholesteryl 1-pyrenebutyrate, cholesteryl-1-pyrenedecanoate, cholesteryl 1-pyrenehexanoate, 22-(N-(7-nitrobenz-2-oxa-1,3-diazol-4-yl)amino)-23,24-bisnor-5-cholen-3β-ol,22-(N-(7-nitrobenz-2-oxa-1,3-diazol-4-yl)amino)-23,24-bisnor-5-cholen-3β-yl cis-9-octadecenoate, 1-pyrenemethyl3-hydroxy-22,23-bisnor-5-cholenate, 1-pyrene-methyl 3β-(cis-9-octadecenoyloxy)-22,23-bisnor-5-cholenate, acridine orange 10-dodecyl bromide, acridine orange 10-nonyl bromide, 4-(N,N-dimethyl-N-tetradecylammonium)-methyl-7-hydroxycoumarin) chloride, 5-dodecanoylaminofluorescein, 5-dodecanoylaminofluorescein-bis-4,5-dimethoxy-2-nitrobenzyl ether, 2-dodecylresorufin, fluorescein octadecyl ester, 4-heptadecyl-7-hydroxycoumarin, 5-hexadecanoylaminoeosin, 5-hexadecanoylaminofluorescein, 5-octadecanoylaminofluorescein, N-octadecyl-N′-(5-(fluoresceinyl))thiourea, octadecyl rhodamine B chloride, 2-(3-(diphenylhexatrienyl)-propanoyl)-1-hexadecanoyl-sn-glycero-3-phosphocholine, 6-N-(7-nitrobenz-2-oxa-1,3-diazol-4-yl)amino)hexanoic acid, 1-hexadecanoyl-2-(1-pyrenedecanoyl)-sn-glycero-3-phosphocholine, 1,1′-dioctadecyl-3,3,3′,3′-tetramethyl-indocarbocyanine perchlorate, 12-(9-anthroyloxy)oleic acid, 5-butyl-4,4-difluoro-4-bora-3a,4a-diaza-s-indacene-3-nonanoic acid, N-(Lissamine™ rhodamine B sulfonyl)-1,2-dihexadecanoyl-sn-glycero-3-phosphoethanolamine, triethylammonium salt, phenylglyoxal monohydrate, naphthalene-2,3-dicarboxaldehyde, 8-bromomethyl-4,4-difluoro-1,3,5,7-tetramethyl-4-bora-3a,4a-diaza-s-indacene, o-phthaldialdehyde, Lissamine™ rhodamine B sulfonyl chloride, 2′,7′-difluorofluorescein, 9-anthronitrile, 1-pyrenesulfonyl chloride, 4-(4-(dihexadecylamino)-styryl)-N-methylpyridinium iodide, chlorins, such as chlorin, chlorin e6, bonellin, mono-L-aspartyl chlorin e6, mesochlorin, mesotetraphenylisobacteriochlorin, and mesotetraphenylbacteriochlorin, hypocrellin B, purpuris, such as octaethylpurpurin, zinc(IV) etiopurpurin, tin(IV) etiopurpurin and tin ethyl etiopurpurin, lutetium texaphyrin, photofrin, metalloporphyrins, protoporphyrin IX, tin protoporphyrin, benzoporphyrin, haematoporphyrin, phthalocyanines, naphthocyanines, merocyanines, lanthanide complexes, silicon phthalocyanine, zinc phthalocyanine, aluminum phthalocyanine, Ge octabutyoxyphthalocyanines, methyl pheophorbide-α-(hexyl-ether), porphycenes, ketochlorins, sulfonated tetraphenylporphines, δ-aminolevulinic acid, texaphyrins, including, for example, 1,2-dinitro-4-hydroxy-5-methoxybenzene, 1,2-dinitro-4-(1-hydroxyhexyl)oxy-5-methoxybenzene, 4-(1-hydroxyhexyl)oxy-5-methoxy-1,2-phenylenediamine, and texaphyrin-metal chelates, including the metals Y(III), Mn(II), Mn(III), Fe(II), Fe(III) and the lanthanide metals Gd(III), Dy(III), Eu(III), La(III), Lu(III) and Tb(III), chlorophyll, carotenoids, flavonoids, bilins, phytochromes, phycobilins, phycoerythrins, phycocyanines, retinoic acids, retinoins, retinates, or combinations of any of the above.

One skilled in the art will readily recognize or can readily determine which of the above compounds are, for example, fluorescent materials and/or photosensitizers. LISSAMINE is the trademark for N-ethyl-N-[4-[[4-[ethyl [(3-sulfophenyl)methyl]amino]phenyl](4-sulfopheny-1)-methylene]-2,5-cyclohexadien-1-ylidene]-3-sulfobenzene-methanaminium hydroxide, inner salt, disodium salt and/or ethyl[4[p[ethyl(m-sulfobenzyl)amino]-o-(p-sulfophenyl)benzylidene]-2,5-cyclohexadien-1-ylidene](m-sulfobenzyl)ammonium hydroxide inner salt disodium salt (commercially available from Molecular Probes, Inc., Eugene, Oreg.). Other suitable photoactive agents for use in the present invention include those described in U.S. Pat. No. 4,935,498, such as a dysprosium complex of 4,5,9,24-tetraethyl-16-(1-hydroxyhexyl)oxy-17 methoxypentaazapentacyclo-(2 0.2.1.1³,6.1⁸,11.0¹⁴,19)-heptacosa-1,3,5,7,9,11(27),12,14,16,18,20,22(25),23-tridecaene and dysprosium complex of 2-cyanoethyl-N,N-diisopropyl-6-(4,5,9,24-tetraethyl-17-methoxypentaazapent acyclo-(20.2.1.1³,6.1⁸,11.0¹⁴,19)-heptacosa-1,3,5,7,9, 11(27), 12,14,16,18,20,22(25),23-tridecaene-16-(1-oxy)hexylphosphoramidite.

Methods of Preparation of the Compostions

The emulsions of the present invention may be prepared by various techniques. In a typical procedure for preparing the emulsions of the invention, the oil coupled to a high Z number atom and the components of the lipid/surfactant coating are fluidized in aqueous medium to form an emulsion. The functional components of the surface layer may be included in the original emulsion, or may later be covalently coupled to the surface layer subsequent to the formation of the nanoparticle emulsion. In one particular instance, for example, where a nucleic acid targeting agent or drug is to be included, the coating may employ a cationic surfactant and the nucleic acid adsorbed to the surface after the particle is formed.

Generally, the emulsifying process involves directing high pressure streams of mixtures containing the aqueous solution, a primer material or the specific binding species, the oil coupled to a high Z number atom and a surfactant (if any) so that they impact one another to produce emulsions of narrow particle size and distribution. The MICROFLUIDIZER apparatus (Microfluidics, Newton, Mass.) can be used to make the preferred emulsions. The apparatus is also useful to post-process emulsions made by sonication or other conventional methods. Feeding a stream of emulsion droplets through the MICROFLUIDIZER apparatus yields formulations small size and narrow particle size distribution.

An alternative method for making the emulsions involves sonication of a mixture of an oil coupled to a high Z number atom and an aqueous solution containing a suitable primer material and/or specific binding species. Generally, these mixtures include a surfactant. Cooling the mixture being emulsified, minimizing the concentration of surfactant, and buffering with a saline buffer will typically maximize both retention of specific binding properties and the coupling capacity of the primer material. These techniques provide excellent emulsions with high activity per unit of absorbed primer material or specific binding species.

When high concentrations of a primer material or specific binding species coated on lipid emulsions, the mixture should be heated during sonication and have a relatively low ionic strength and moderate to low pH. Too low an ionic strength, too low a pH or too much heat may cause some degradation or loss of all of the useful binding properties of the specific binding species or the coupling capacity of the primer material. Careful control and variation of the emulsification conditions can optimize the properties of the primer material or the specific binding species while obtaining high concentrations of coating. Prior to administration, these formations may be rendered sterile with techniques known in the art, for example, terminal steam sterilization.

The emulsion particle sizes can be controlled and varied by modification of the emulsification techniques and the chemical components. Techniques and equipment for determining particle sizes are known in the art and include, but not limited to, laser light scattering and an analyzer for determining laser light scattering by particles.

When appropriately prepared, the nanoparticles that comprise ancillary agents contain a multiplicity of functional such agents at their outer surface, the nanoparticles typically contain hundreds or thousands of molecules of the biologically active agent, targeting ligand, radionuclide, MRI contrast agent and/or PET contrast agent. For MRI contrast agents, the number of copies of a component to be coupled to the nanoparticle is typically in excess of 5,000 copies per particle, more preferably 10,000 copies per particle, still more preferably 30,000, and still more preferably 50,000-100,000 or more copies per particle. The number of targeting agents per particle is typically less, of the order of several hundred while the concentration of PET contrast agents, fluorophores, radionuclides, and biologically active agents is also variable.

The nanoparticles need not contain an ancillary agent. In general, because the particles have a high Z number atom oil core, X-ray imaging and, in some cases, ultrasound imaging can be used to track the location of the particles concomitantly with any additional functions described herein. Additionally, such particles coupled to a targeting ligand are particularly useful themselves as imaging contrast agents. Further, the inclusion of other components in multiple copies renders them useful in other respects as described herein. For instance, the inclusion of a chelating agent containing a paramagnetic ion makes the emulsion useful as an MRI contrast agent. The inclusion of biologically active materials makes them useful as drug delivery systems. The inclusion of radionuclides makes them useful either as therapeutic for radiation treatment or as diagnostics for imaging. Other imaging agents include fluorophores, such as fluorescein or dansyl. Biologically active agents may be included. A multiplicity of such activities may be included; thus, images can be obtained of targeted tissues at the same time active substances are delivered to them.

The emulsions can be prepared in a range of methods depending on the nature of the components to be included in the coating. In a typical procedure, used for illustrative purposes only, the following procedure is set forth: Ethiodol (iodized oil, 20% w/v), a surfactant co-mixture (2.0%, w/v), glycerin (1.7%, w/v) and water representing the balance is prepared where the surfactant co-mixture includes 70 mole % lecithin, 28 mole % cholesterol and 2 mole % dipalmitoyl-phosphatidylethanolamine (DPPE) dissolved in chloroform. A drug is added in titrated amounts between 0.01 and 50 mole % of the 2% surfactant layer, between 0.01 and 20 mole % of the 2% surfactant layer, between 0.01 and 10 mole % of the 2% surfactant layer, between 0.01 and 5.0 mole % of the 2% surfactant layer, preferably between 0.2 and 2.0 mole % of the 2% surfactant layer. The chloroform-lipid mixture is evaporated under reduced pressure, dried in a 50° C. vacuum oven overnight and dispersed into water by sonication. The suspension is transferred into a blender cup (for example, from Dynamics Corporation of America) with iodized oil in distilled or deionized water and emulsified for 30 to 60 seconds. The emulsified mixture is transferred to a Microfluidics emulsifier and continuously processed at 20,000 PSI for four minutes. The completed emulsion is vialed, blanketed with nitrogen and sealed with stopper crimp seal until use. A control emulsion can be prepared identically excluding the drug from the surfactant co-mixture. Particle sizes are determined in triplicate at 37° C. with a laser light scattering submicron particle size analyzer (Malvern Zetasizer 4, Malvern Instruments Ltd., Southborough, Mass.), which indicate tight and highly reproducible size distribution with average diameters less than 200 nm. Unincorporated drug can be removed by dialysis or ultrafiltration techniques. To provide the targeting ligand, for example, an antibody or antibody fragment or a non-peptide ligand is coupled covalently to the phosphatidyl ethanolamine through a bifunctional linker in the procedure described herein.

Kits

The emulsions of the invention may be prepared and used directly in the methods of the invention, or the components of the emulsions may be supplied in the form of kits. The kits may comprise the untargeted composition containing all of the desired ancillary materials in buffer or in lyophilized form. The kits may comprise the pre-prepared targeted composition containing all of the desired ancillary materials and targeting materials in buffer or in lyophilized form. Alternatively, the kits may include a form of the emulsion which lacks the targeting agent which is supplied separately. Under these circumstances, typically, the emulsion will contain a reactive group, such as a maleimide group, which, when the emulsion is mixed with the targeting agent, effects the binding of the targeting agent to the emulsion itself. A separate container may also provide additional reagents useful in effecting the coupling. Alternatively, the emulsion may contain reactive groups which bind to linkers coupled to the desired component to be supplied separately which itself contains a reactive group. A wide variety of approaches to constructing an appropriate kit may be envisioned. Individual components which make up the ultimate emulsion may thus be supplied in separate containers, or the kit may simply contain reagents for combination with other materials which are provided separately from the kit itself.

A non-exhaustive list of combinations might include: emulsion preparations that contain, in their lipid-surfactant layer, an ancillary component such as a fluorophore or chelating agent and reactive moieties for coupling to the targeting agent; the converse where the emulsion is coupled to targeting agent and contains reactive groups for coupling to an ancillary material; emulsions which contain both targeting agent and a chelating agent but wherein the metal to be chelated is either supplied in the kit or independently provided by the user; preparations of the nanoparticles comprising the surfactant/lipid layer where the materials in the lipid layer contain different reactive groups, one set of reactive groups for a targeted ligand and another set of reactive groups for an ancillary agent; preparation of emulsions containing any of the foregoing combinations where the reactive groups are supplied by a linking agent.

Methods of Use of the Compositions

The emulsions and kits for their preparation are useful in the methods of the invention which include imaging of cells, tissues and/or organs, and/or delivery of therapeutic agents to the cells, tissues and/or organs. In some embodiments, the emulsions are targeted to a particular cell type and/or tissue through the use of ligands directed to the cell and/or tissue on the surface of the emulsions. The emulsions can be used with cells or tissues in vivo, ex vivo, in situ and in vitro.

In vitro or ex vivo use of the emulsions containing a targeting ligand and an agent (e.g., drug) can, for example, identify and/or deliver the agent to the targeted cell. Such cells can be identified using X-ray imaging techniques, for example, and agent delivery to the cell can also be confirmed through the imaging process. For example, the targeted emulsions can be used to deliver genetic material to cells, e.g., stem cells, and/or to label cells, e.g., stem cells, ex vivo or in vitro before implantation or further use of the cells. The presence of the high Z number atoms in the particulate emulsions often results in emulsions that are typically heavier than water. Accordingly, the emulsions of the invention can be used to identify targeted cells in solution and to collect or isolate targeted cells from a solution, for example, by precipitation and/or gradient centrifuguation.

The methods of using the nanoparticulate emulsions of the invention in vivo and in vitro are well known to those in the art. Cardiovascular-related tissues, for example, are of interest to be imaged and/or treated using the emulsions of the invention, including, but limited to, heart tissue and all cardiovascular vessels, angiogenic tissue, any part of a cardiovascular vessel, any material or cell that comes into or caps cardiovascular a vessel, e.g., thrombi, clot or ruptured clot, platelets, muscle cells and the like. Disease conditions to be imaged and/or treated using the emulsions of the invention include, but are not limited to, any disease condition in which vasculature plays an important part in pathology, for example, cardiovascular disease, cancer, areas of inflammation, which may characterize a variety of disorders including rheumatoid arthritis, areas of irritation such as those affected by angioplasty resulting in restenosis, tumors, and areas affected by atherosclerosis. Depending upon the targeting ligand used, emulsions of the invention are of particular use in vascular and/or restenosis imaging. For example, emulsions containing a ligand that bind to αᵥβ₃ integrin are targeted to tissues containing high expression levels of αᵥβ₃ integrin. High expression levels of αᵥβ₃ are typical of activated endothelial cells and are considered diagnostic for neovasculature. Other tissues of interest to be imaged and/or treated include those containing particular malignant tissue and/or tumors.

The combination of target-directed imaging and therapeutic agent delivery allows both the identification of a target and the agent delivery in a single procedure, if desired. The ability to image the emulsions delivering the agent provides for identification and/or confirmation of the cells or tissue to which the agent is delivered.

In addition to combining imaging with therapeutic agent delivery, emulsions of the invention can be used in single-modal or multi-modal imaging. For example, multi-modal imaging can be performed with emulsions including ancillary reagents that allow for more than one type of imaging such as the combination of X-ray and MRI imaging or other combinations of the types of imaging described herein.

For use as X-ray contrast agents, the compositions of the present invention generally have an oil coupled to a high Z number atom concentration of about 10% to about 60% w/v, preferably of about 15% to about 50% w/v, more preferably between about 20% to about 40%. Generally, elements with higher Z number can be used in lower concentrations than elements with lower Z numbers. Dosages, administered by intravenous injection, will typically range from 0.5 mmol/kg to 1.5 mmol/kg, preferably 0.8 mmol/kg to 1.2 mmol/kg. Imaging is performed using known techniques, preferably X-ray computed tomography.

The ultrasound contrast agents of the present invention are administered, for example, by intravenous injection by infusion at a rate of approximately 3 μL/kg/min. Imaging is performed using known techniques of sonography.

The magnetic resonance imaging contrast agents of the present invention may be used in a similar manner as other MRI agents as described in U.S. Pat. Nos. 5,155,215 and 5,087,440; Margerstadt et al. (1986) Magn. Reson. Med. 3:808; Runge et al (1988) Radiology 166:835; and Bousquet et al. (1988) Radiology 166:693. Other agents that may be employed are those set forth in U.S. Pat. publication 2002/0127182 which are pH sensitive and can change the contrast properties dependent on pulse. Generally, sterile aqueous solutions of the contrast agents are administered to a patient intravenously in dosages ranging from 0.01 to 1.0 mmoles per kg body weight.

The diagnostic radiopharmaceuticals are administered by intravenous injection, usually in saline solution, at a dose of 1 to 100 mCi per 70 kg body weight, or preferably at a dose of 5 to 50 mCi. Imaging is performed using known procedures.

The therapeutic radiopharmaceuticals are administered, for example, by intravenous injection, usually in saline solution, at a dose of 0.01 to 5 mCi per kg body weight, or preferably at a dose of 0.1 to 4 mCi per kg body weight. For comparable therapeutic radiopharmaceuticals, current clinical practice sets dosage ranges from 0.3 to 0.4 mCi/kg for Zevalin™ to 1-2 mCi/kg for OctreoTher™, a labeled somatostatin peptide. For such therapeutic radiopharmaceuticals, there is a balance between tumor cell kill vs. normal organ toxicity, especially radiation nephritis. At these levels, the balance generally favors the tumor cell effect. These dosages are higher than corresponding imaging isotopes.

As used herein, an “individual” is a vertebrate, preferably a mammal, more preferably a human. Mammals include, but are not limited to, humans, farm animals, sport animals, rodents and pets.

As used herein, an “effective amount” or a “sufficient amount” of a substance is that amount sufficient to effect beneficial or desired results, including clinical results, and, as such, an “effective amount” depends upon the context in which it is being applied. An effective amount can be administered in one or more administrations.

As used herein, the singular form “a”, “an”, and “the” includes plural references unless indicated otherwise. For example, “a” target cell includes one or more target cells.

The following Examples are offered to illustrate but not to limit the invention.

### EXAMPLES

The following examples illustrate that targeting of the nanoparticles may be accomplished by directly or indirectly coupling homing ligands to the surface of the nanoparticles with the same net effect from the bound particles. The homing ligands may be added before or after the emulsion particles are made.

### Example 1

### Preparation of Biotinylated Targeted X-Ray Contrast Agents

A biotinylated x-ray contrast agent was produced by incorporating biotinylated phosphatidylethanolamine (Avanti Polar Lipids, Alabaster, Ala.) into the outer lipid monolayer of an iodized oil emulsion. A 2% (w/v) lipid surfactant co-mixture included lecithin (70 mole %, Pharmacia Inc., Clayton, N.C.), cholesterol (28 mole %, Sigma Chemical Co., St. Louis, Mo.), and biotin-caproate-phosphatidylethanolamine (2 mol %), which were dissolved in chloroform, evaporated under reduced pressure, dried in a 50° C. vacuum oven, and dispersed into water by sonication. The suspension was combined with iodized oil (Ethiodol, Savage Laboratories, Melville, N.Y.), distilled, deionized water and was continuously processed at 20,000 PSI for 4 minutes with an S110 Microfluidics emulsifier (Microfluidics, Newton, Mass.). A control agent was prepared by substituting unmodified phosphatidylethanolamine for the biotinylated form. Particle sizes were determined in triplicate at 37° C. to be nominally less than 200 nm for the treated and control emulsions using a laser light scattering submicron particle size analyzer (Malvern Instruments, Malvern, Worcestershire, UK).

### Example 2

### Preparation of Targeted Contrast Agents Using Directly Conjugated Ligands Coupled Before Emulsification

The nanoparticulate emulsions are comprised of 20% (w/v) iodized oil (Ethiodol, Savage Laboratories), 2% (w/v) of a surfactant co-mixture, 1.7% (w/v) glycerin and water representing the balance. The surfactant of control, i.e. non-targeted, nanoemulsions, included 70 mole % lecithin (Avanti Polar Lipids, Inc.), 28 mole % cholesterol (Sigma Chemical Co.), 2 mole % dipalmitoyl-phosphatidylethanolamine (DPPE) (Avanti Polar Lipids, Inc.). αᵥβ₃-targeted CT nanoparticles are prepared as above with a surfactant co-mixture that included: 70 mole % lecithin, 0.05 mole % N-[{w-[4-(p-maleimidophenyl) butanoyl]amino}poly(ethylene glycol)2000] 1,2-distearoyl-sn-glycero-3-phosphoethanolamine (MPB-PEG-DSPE) covalently coupled to the αᵥβ₃-integrin peptidomimetic antagonist (Bristol-Myers Squibb Medical Imaging, Inc., North Billerica, Mass.), 28 mole % cholesterol, and 1.95 mole % DPPE. The components for each nanoparticle formulation are emulsified in a M110S Microfluidics emulsifier (Microfluidics) at 20,000 PSI for four minutes. The completed emulsions were placed in crimp-sealed vials and blanketed with nitrogen. Particle sizes are determined at 37° C. with a laser light scattering submicron particle size analyzer (Malvern Instruments).

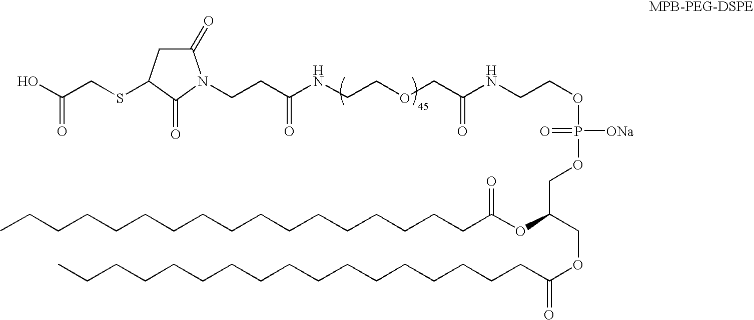

A peptidomimetic or small peptide modified for use with the addition of an available thiol group, e.g., a peptide spacer terminated with mercaptoacetic acid, is coupled to a phosphatidylethanolamine through a PEG₍₂₀₀₀₎ maleimide spacer (MPB-PEG-DSPE). MPB-PEG-DSPE is combined at a 1:1 molar ratio with the mimetic or small peptide in 3 ml of N₂-purged, 6 mM EDTA. The round bottom flask is then mildly sonicated in a water bath for 30 minutes under a slow stream of N₂ at 37°-40° C. The mixture is swirled occasionally to resuspend all of the lipid film. This premix is added to the remaining surfactant components, PFC and water for emulsification.

Alternatively, a solution based coupling process may be used. The process has two parts. In step A, 1,2-distearoyl-sn-glycero-3-phosphoethanolamine-N-[maleimide(polyethylene glycol)2000] is dissolved in DMF and sparged with inert gas (i.e., nitrogen or argon). The oxygen-free solution is adjusted to pH 7-8 using DIEA and treated with mercaptoacetic acid. Stirring is continued at ambient temperatures until consumption of starting materials is complete. The solution is used directly in the following reaction (step B).

In step B, the product solution of step A, above, is pre-activated by the addition of HBTU and sufficient DIEA to maintain pH 8-9. To the solution is added the mimetic or small peptide with an available amino group, and the solution is stirred at room temperature under nitrogen for 18 h. DMF is removed in vacuo and the crude product is purified by preparative HPLC.

### Example 3

### Preparation of Targeted Contrast Agents Using Directly Conjugated Ligands Coupled After Emulsification

The nanoparticulate emulsions are comprised of 20% (w/v) iodized oil (Ethiodol, Savage Laboratories), 2% (w/v) of a surfactant co-mixture, 1.7% (w/v) glycerin and water representing the balance. The surfactant of control, i.e. non-targeted, emulsions included 70 mole % lecithin (Avanti Polar Lipids, Inc.), 28 mole % cholesterol (Sigma Chemical Co.), 2 mole % dipalmitoyl-phosphatidylethanolamine (DPPE) (Avanti Polar Lipids, Inc.). Targeted CT nanoparticles are prepared as above with a surfactant co-mixture that included: 70 mole % lecithin, 0.05 mole % N-[{w-[4-(p-maleimidophenyl) butanoyl]amino}poly(ethylene glycol)2000]1,2-distearoyl-sn-glycero-3-phosphoethanolamine (MPB-PEG-DSPE), 28 mole % cholesterol, and 1.95 mole % DPPE. The components for each nanoparticle formulation are emulsified in a M110S Microfluidics emulsifier (Microfluidics) at 20,000 PSI for four minutes. The completed emulsions are placed in crimp-sealed vials and blanketed with nitrogen until coupled. Particle sizes are determined at 37° C. with a laser light scattering submicron particle size analyzer (Malvern Instruments).

A free thiol containing ligand (e.g., antibody or antibody fragment) is dissolved in deoxygenated 50 mM sodium phosphate, 10 mM EDTA pH 6.65 buffer at a concentration of approx. 10 mg/ml. This solution is added, under nitrogen, to the nanoparticles in an equimolar ratio of the MPB-PEG₍₂₀₀₀₎-DSPE contained in the surfactant to ligand. The vial is sealed under nitrogen (or other inert gas) and allowed to react at ambient temperature with gentle agitation for a period of 4 to 16 hours. Excess (i.e., unbound) ligand may be dialyzed against phosphate/EDTA buffer using a Spectra/Por “Dispodialyzer”, 300,000 MWCO (Spectrum Laboratories, Rancho Dominguez, Calif.), if required.

### Example 4

### Use of Targeted X-Ray Contrast Agent Directed Against Fibrin In Vitro and Imaged with CT

Part 1: Preparation and In Vitro Targeting of Fibrin-Rich Clots

To prepare fibrin-rich clots, citrated plasma (375 uL), calcium chloride (22 uL 500 mM) and thrombin (3U) were combined in a plastic tubular mold through which a 4-0 polyester suture is passed. Formation of bubbles was avoided. The fibrin clot formed quickly around and attached to the suture. A hole was placed through the cap and bottom of a 12×75 mm polyethylene snap cap tube. The clot was removed from the mold and positioned within the tube with the suture passing out through the holes at the top and bottom. The holes in the tube were sealed with hot glue and tube was filled with saline.

Eight (8) clots were prepared, incubated at 4° C. overnight with 125 ug of biotinylated 1H10 anti-fibrin antibody, rinsed three (3) times with phosphate buffered saline, then exposed with 125 ug of avidin at 37° C. for 1 hour. Excess avidin was rinsed away with three changes of phosphate buffered saline. Clots were treated with the non-targeted (n=4, nonbiotinylated) or targeted (n=4, biotinylated) x-ray contrast agent prepared as described in Example 1 for 1 hour at 37° C. Unbound nanoparticles were washed from clots with three exchanges of phosphate buffer.

Part 2: Imaging of Targeted Clots with Computer Tomography

Clots within the tubes were positioned with the bore of a Philips AcQSim-CT scanner and imaged with the following specifications:
Slice Thickness: 3.0 mm
KVP [Peak Output, KV]: 80.0
FOV: 480.0 mm
Spatial Resolution: 1.0 mm
Distance Source to Detector [mm]: 1498.350
Distance Source to Patient [mm]: 635.35
Exposure Time [ms]: 808727348
X-ray Tube Current [mA]: 400
Rows: 512
Columns: 512
Pixel Spacing: 0.1562500\0.1562500
Pixel Aspect Ratio: 1\1.

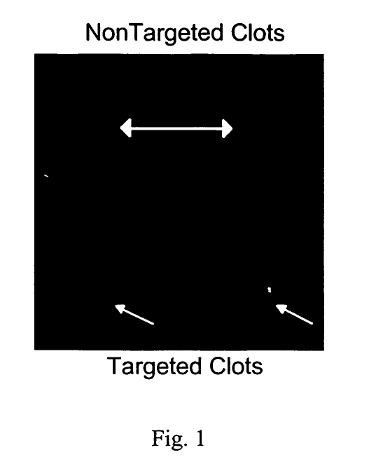

Examples of fibrin clot images are shown in FIG. 1. FIG. 1 shows two examples of fibrin clots exposed to the nontargeted (top) and targeted (below) contrast agents. Targeted x-ray nanoparticles bound to the surface of the fibrin clot to provide contrast enhancement around the thrombus perimeter, which clearly delineates surface shape (in cross-section) and distinguishes the clot from surrounding saline background. No contrast enhancement is appreciated within the clot core because the nanoparticles are sterically excluded by dense fibrin packing. The nontargeted fibrin-rich clots reveal no peripheral x-ray contrast enhancement and are difficult to distinguish from the surrounding saline background.

The contrast to noise ratio (CNR) of the imaged clots was computed as the signal of the clot surface minus the signal from the surrounding saline media all divided by the standard deviation of the surrounding saline signal. The targeted x-ray nanoparticles provided a CNR of 22.1 as compared to the baseline (non-targeted) control clots which had a CNR of 5.0. Thus, use of the targeted nanoparticles resulted in a 400% improvement in CNR. These results demonstrate that targeted x-ray nanoparticles, regardless of the targeting method, provide enhanced x-ray contrast enhancement.

1. An oil-in-water emulsion comprising nanoparticles formed from an oil-like compound coupled to an atom with a Z number above 36, wherein said nanoparticles are coated with a lipid/surfactant layer and wherein said nanoparticles are coupled to a ligand which binds to a target.

2. The emulsion of claim 1, wherein said atom with a Z number above 36 is covalently coupled to the oil-like compound.

3. The emulsion of claim 1, wherein said atom with a Z number above 36 is selected from the group consisting of yttrium, zirconium, silver, tin, iodine, barium, tantalum, platinum, gold, and bismuth.

4. The emulsion of claim 1, wherein said nanoparticles further include at least one magnetic resonance imaging (MRI) contrast agent.

5. The emulsion of claim 4, wherein said MRI contrast agent is a metal ion.

6. The emulsion of claim 5, wherein said MRI contrast agent is a chelated paramagnetic ion.

7. The emulsion of claim 6, wherein said chelating agent is MEO-DOTA and said paramagnetic ion is gadolinium ion.

8. The emulsion of claim 1, wherein said nanoparticles further include at least one biologically active agent.

9. The emulsion of claim 8, wherein said biologically active agent is a hormone or pharmaceutical agent.

10. The emulsion of claim 1, wherein said nanoparticles further contain at least one radionuclide.

11. The emulsion of claim 10, wherein said radionuclide is ^{99m}Tc.

12. The emulsion of claim 1, wherein said nanoparticles further include at least one fluorophore.

13. The emulsion of claim 12, wherein said fluorophore is fluorescein.

14. The emulsion of claim 1, wherein said ligand comprises a biotin agent or an avidin agent.

15. The emulsion of claim 1, wherein said ligand is an antibody, fragment of an antibody, a non-peptide ligand, a polypeptide, a polysaccharide, an aptmer, a lipid, a nucleic acid or a lectin.

16. A method to deliver a bioactive agent to a target tissue, comprising administering an emulsion according to claim 8 to an individual comprising said target tissue.

17. The method according to claim 16, further comprising obtaining an image of said target tissue.

18. A method for imaging a target tissue, comprising administering to the tissue a composition according to claim 1 and obtaining an image of said target tissue.

19. The method according to claim 18, wherein said target tissue is cardiovascular-related tissue.

20. The method according to claim 18, wherein said image is an X-ray image.

21. A method for imaging a target tissue, comprising administering to said tissue a composition according to claim 4 and obtaining a magnetic resonance image of said target tissue.

22. A method for imaging a target tissue, comprising administering to said tissue a composition according to claim 12 and obtaining an image of said target tissue bound to the fluorophore.
